(19) United States
(12) Patent Application Publication
(10) Pub. No.: US 2023/0241161 A1
(43) Pub. Date: Aug. 3, 2023

(54) RSPO1 PROTEINS AND THEIR USE

(71) Applicants: INSERM (INSTITUTNATIONALDELASANTEETDELARECHERCHEMEDICALE), Paris (FR); CENTRE NATIONAL DELARECHERCHESCIENTIFIQUE-CNRS-, Paris (FR); UNIVERSITE COTE D’AZUR

(72) Inventors: Patrick COLLOMBAT, Tourrette-Levens (FR); Serena SILVANO, Nice (FR)

(21) Appl. No.: 17/758,540

(22) PCT Filed: Jan. 8, 2021
(86) PCT No.: PCT/EP2021/050289

§ 371 Date: Jul. 8, 2022

(30) Foreign Application Priority Data
Jan. 10, 2020 (EP) 20305016.6

(51) Int. Cl. A61K 38/17; A61P 3/10

(52) U.S. Cl. CPC A61K 38/1709; A61P 3/10

(57) ABSTRACT

The disclosure relates to Rspo 1 proteins and their use as a medicament, in particular for the treatment of diabetes, such as diabete type 1 or 2. The disclosure also relates to methods for inducing the proliferation of pancreatic beta cells in a human subject in need thereof, said method comprising administering an efficient amount of an Rspo 1 protein in said subject. Inventors have surprisingly shown that treatments with recombinant Rspo1 protein induce in vivo proliferation of functional pancreatic beta cells, and improve glucose tolerance and increase glucose-stimulated insulin secretion (GSIS) in mice models of diabete. In addition, they found out that upon near complete beta-cell ablation, the remaining beta-cells could be induced with Rspo 1 protein administration to proliferate and reconstitute a functional beta-cells mass able to maintain euglycemia. Lastly, they showed that Rspo1 can also induce human beta-cell proliferation opening new unexpected avenues for the treatment and prevention of diabetes in human.

The disclosure relates to Rspo1 proteins for their use as a medicament, in particular for the treatment of diabetes.

### BACKGROUND

Over the past few decades, diabetes has become one of the most widespread metabolic disorders with an epidemic dimension affecting almost 9% of the world’s population (WHO, 2016). By the year 2049, the number of people affected by diabetes is projected to reach 600 million. Diabetes is characterized by high blood glucose levels, which, in most cases, result from the inability of the pancreas to secrete sufficient amounts of insulin. While type 1 diabetes (T1D) is caused by the autoimmune-mediated destruction of insulin-producing β-cells, type 2 diabetes (T2D) results from a resistance to insulin action and an eventual β-cell failure/loss over time.

Current treatments of diabetes fail to strictly restore normoglycemia and, in the case of T1D, even appear as rather palliative, replacing defective insulin secretion by exogenous insulin injections. Therefore, replenishing the pancreas with new functioning P-cells and/or maintaining the health of the remaining P-cells represent key strategies for the treatment of both conditions. However, to date, there is no available treatments preventing the loss of, or inducing the proliferation of pancreatic beta cells, especially in human patients suffering from diabete type 1.

Rspo1 belong to a family of cysteine-rich secreted proteins, including also Rspo2, Rspo3 and Rspo4. They share a common structural architecture, including four structurally and functionally different domains. At the N-terminal, a signal peptide sequence ensures the correct entry of R-spondin proteins in the canonical secretory pathway. The mature secreted form contains two amino-terminal cysteine-rich furin-like repeats (FU1 and FU2), crucial for the interaction with R-spondin-specific receptors LGR (Leucine-rich repeat-containing G-protein coupled receptor) 4-6 (de Lau, W. B., Snel, B. & Clevers, H. C. Genome Biol 13, 242, doi:10.1186/gb-2012-13-3-242 (2012)). The central part of the protein contains one thrombospondin type-1 repeat domain (TSP1), involved in the interactions with specific components of the extracellular matrix, followed by a carboxy-terminal basic-amino acid rich domain, whose role has not yet been clarified. R-spondin proteins were reported to exert a key role in processes, such as cell proliferation (Kim, K. A. et al. Science 309, 1256-1259, doi:10.1126/science.1112521 (2005). Da Silva, F. et al. Dev Biol 441, 42-51, doi:10.1016/j.ydbio.2018.05.024 (2018)), cell specification (Vidal, V. et al. Genes Dev 30, 1389-1394, doi:10.1101/gad.277756.116 (2016)) and sex determination (Chassot, A. A. et al. Hum Mol Genet 17, 1264-1277, doi:10.1093/hmg/ddn016 (2008)) and they have been reported as central regulators of the canonical WNT signaling pathway (also known as WNT/β-catenin or cWNT pathway) (Jin, Y. R. & Yoon, J. K. The R-spondin family of proteins: emerging regulators of WNT signaling. Int J Biochem Cell Biol 44, 2278-2287, doi:10.1016/j.biocel.2012.09.006 (2012)).

Despite the great deal of interest raised by the possible involvement of the cWNT pathway in pancreas maturation and function (Scheibner et al 2019, Curr Opin Cell Biol. 61 :48-55), the roles and the contribution of R-spondin proteins have been poorly investigated in this organ.

In vitro analyses reported that, in the presence of Rspo1, β-cell proliferation and function are increased in the Min6 tumor-derived cell line (Wong, V. S., Yeung, A., Schultz, W. & Brubaker, P. L. R-spondin-1 is a novel beta-cell growth factor and insulin secretagogue. J Biol Chem 285, 21292-21302, doi:10.1074/jbc.M110.129874 (2010)). However, further more recent studies from the same group reported contradictory statements : Rspo1 deficiency in mice is associated with increased β-cell mass and enhanced glycemic controls (Wong, V. S., Oh, A. H., Chassot, A. A., Chaboissier, M. C. & Brubaker, P. L. Diabetologia 54, 1726-1734, doi:10.1007/s00125-011-2136-2 (2011) and Chahal et al 201, Pancreas Vol 43(1) pp 93-102).

In contrast to the latter studies, the inventors have now surprisingly shown that treatments with recombinant Rspo1 protein induce in vivo proliferation of functional pancreatic beta cells, and improve glucose tolerance and increase glucose-stimulated insulin secretion (GSIS) in mice models of diabete. In addition, they found out that upon near complete beta-cell ablation, the remaining beta-cells could be induced with Rspo1 protein administration to proliferate and reconstitute a functional beta-cells mass able to maintain euglycemia. Lastly, they showed that Rspo1 can also induce human beta-cell proliferation opening new unexpected avenues for the treatment and prevention of diabetes in human.

### SUMMARY

The present disclosure relates to isolated Rspo1 proteins and their use as a medicament, preferably in the treatment of diabetes in a subject in need thereof.

In specific embodiments, said Rspo1 protein of the disclosure, is either
(i) a protein comprising a Rspondin-1 polypeptide,
(ii) a protein comprising a functional fragment of Rspondin-1 polypeptide, or
(iii) a protein comprising a functional variant of Rspondin-1 polypeptide.

In specific embodiments, said Rspo1 protein of the disclosure is either
(i) a protein comprising a human Rspondin1 polypeptide of any one of SEQ ID NOs:2-4,
(ii) a protein comprising a functional fragment of human Rspondin-1 polypeptide of any one of SEQ ID NOs:2-4, or
(iii) a protein comprising a functional variant of Rspondin-1 polypeptide of any one of SEQ ID NOs:2-4.

In specific embodiments, said Rspo1 protein of the disclosure is a protein comprising a functional fragment of Rspondin-1 polypeptide, said functional fragment preferably comprising or consisting of a polypeptide having at least 40-100 consecutive amino acid residues in the FU1 and/or FU2 domains of Rspondin1 protein, typically at least 40-100 consecutive amino acid residues of any of the polypeptides of SEQ ID NO: 1-4 and SEQ ID NO:8-24.

In specific embodiments, said Rspo1 protein of the disclosure is a recombinant protein comprising either
(i) any one of SEQ ID NO: 1-4 and SEQ ID NO:8-24, or
(ii) a combination of fragments of Rspo1 protein of SEQ ID NO:1, typically including the functional domain FU1 and the functional domain FU2, and, optionally the functional domain TSP.

In specific embodiments, said Rspo1 protein of the disclosure binds to LGR4 receptor.

In specific embodiments, said Rspo1 protein of the disclosure induces the proliferation of functional beta cells as determined in an in vitro beta cell proliferation assay and/or in an in vivo beta cell proliferation assay.

In specific embodiments, said Rspo1 protein of the disclosure is a protein comprising a functional fragment or functional variant of a native Rspondin-1 polypeptide preferably, of human R-spondin-1 of SEQ ID NO :3 or 4, and said Rspo1 protein exhibits at least 50%, 60%, 70%, 80%, 90% 100% or more of one or more of the following activities relative to said native R-spondin 1:
(i) Binding affinity to LGR4 receptor, for example as determined by SPR assay ;
(ii) Induction of the proliferation of functional beta cells, for example as determined in an in vitro beta cell proliferation assay;
(iii) Induction of the proliferation of functional beta cells, for example as determined in an in vivo beta cell proliferation assay;
(iv) Increase of glucose-stimulated insulin secretion (GSIS), for example as determined in an in vitro beta cell proliferation assay; or,
(v) Increase of glucose-stimulated insulin secretion (GSIS), for example as determined in an in vivo beta cell proliferation assay.

The above functional assays are for example described in more details in the Examples below.

In specific embodiments, said Rspo1 protein of the disclosure is a protein comprising a functional variant of R-spondin 1, wherein said functional variant comprises or essentially consists of a polypeptide having at least 70%, 80%, 90% or at least 95% identity to a native R-spondin 1 polypeptide sequence, preferably at least 70%, 80%, 90% or at least 95% identity to one of polypeptides of SEQ ID NOs :1-4 and SEQ ID NO :8-24.

In specific embodiments, said functional variant of R-spondin 1 differs from the corresponding native R-spondin 1 sequence through only amino acid substitutions.

In specific embodiments, said Rspo1 protein of the disclosure is a fusion protein, for example a fusion protein comprising an Fc region of an antibody.

In specific embodiments, said Rspo1 protein of the disclosure is a pegylated or PASylated protein.

According to the present disclosure, said Rspo1 proteins are particularly useful in the treatment of diabete type 1 or type 2 and/or in inducing in vivo the proliferation of beta cells and the increase of mass of islets of Langerhans. In specific embodiments, a therapeutically efficient amount of Rspo1 protein is administered via the subcutaneous or intravenous route to a subject in need thereof.

In specific embodiments of such in vivo use of the Rspo1 proteins, said subject is a human subject.

The disclosure also relates to a pharmaceutical composition comprising the Rspo1 protein as defined above, and one or more pharmaceutically acceptable excipients.

In specific embodiments, said pharmaceutical composition further comprises one or more additional pharmaceutical ingredients for treating or preventing diabete, typically, selected from the group consisting of cytokines, anti-viral, anti-inflammatory agents, anti-diabetic or hypoglycemiant agents, cell therapy product (e.g beta cell composition) and immune modulators.

The disclosure also relates to the use of a Rspo1 protein or an analogue as defined herein, in an in vitro method for inducing the proliferation of beta cells, typically human beta cells.

Typically, said in vitro method comprises the following:
(i) providing induced pluripotent stem cells (iPSCs), preferably iPSCs from human cells,
(ii) in vitro differentiating said iPSCs to β-cells of islets of Langerhans, and
(iii) culturing said differentiated β-cells under proliferating conditions,
(iv) wherein a sufficient amount of said Rspo1 protein or analogue is added at step (ii) and/or (iii) for differentiating iPS cells and/or inducing the proliferation of said β-cells.

### DETAILED DESCRIPTION

### Definitions

In order that the present disclosure may be more readily understood, certain terms are first defined. Additional definitions are set forth throughout the detailed description.

The term “amino acid” refers to naturally occurring and unnatural amino acids (also referred to herein as “non-naturally occurring amino acids”), e.g., amino acid analogues and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogues refer to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogues can have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that function similarly to a naturally occurring amino acid. The terms “amino acid” and “amino acid residue” are used interchangeably throughout.

Substitution refers to the replacement of a naturally occurring amino acid either with another naturally occurring amino acid or with an unnatural amino acid. For example, during chemical synthesis of a synthetic peptide, the native amino acid can be readily replaced by another naturally occurring amino acid or an unnatural amino acid.

As used herein, the term “protein” refers to any organic compounds made of amino acids arranged in one or more linear chains (also referred as “polypeptide chains”) and folded into a globular form. It includes proteinaceous materials or fusion proteins. The amino acids in such polypeptide chain may be joined together by the peptide bonds between the carboxyl and amino groups of adjacent amino acid residues. The term “protein” further includes, without limitation, peptides, single chain polypeptide or any complex proteins consisting primarily of two or more chains of amino acids. It further includes, without limitation, glycoproteins or other known post-translational modifications. It further includes known natural or artificial chemical modifications of natural proteins, such as without limitation, glycoengineering, pegylation, hesylation, PASylation and the like, incorporation of non-natural amino acids, amino acid modification for chemical conjugation or other molecule, etc...

The term “recombinant protein”, as used herein, includes proteins that are prepared, expressed, created or isolated by recombinant means, such as fusion proteins isolated from a host cell transformed to express the corresponding protein, e.g., from a transfectoma, etc...

As used herein, the term “fusion protein” refers to a recombinant protein comprising at least one polypeptide chain which is obtained or obtainable by genetic fusion, for example by genetic fusion of at least two gene fragments encoding separate functional domains of distinct proteins. A protein fusion of the present disclosure thus includes at least one of Rspondin-1 polypeptide or a fragment or variant thereof as described below, and at least one other moiety, the other moiety being a polypeptide other than a Rspondin-1 polypeptide or functional variant or fragment thereof. In certain embodiments, the other moiety may also be a non protein moiety, such as, for example, a polyethyleneglycol (PEG) moiety or other chemical moiety or conjugates. The second moiety can be a Fc region of an antibody, and such fusion protein is therefore referred as a « Fc fusion protein ».

As used herein, the term “Fc region” is used to define the C-terminal region of an immunoglobulin heavy chain, including native sequence Fc region and variant Fc regions, preferably containing no more than 5, 10, 15, or 20 insertions, deletions, or substitutions of amino acid relative to the native human Fc region. The native human Fc region can be any of the IgG1, IgG2, IgG3, IgG4, IgA, IgA, IgD, IgE or IgM isotype. The human IgG heavy chain Fc region is generally defined as comprising the amino acid residue from position C226 or from P230 to the carboxyl-terminus of the IgG antibody. The numbering of residues in the Fc region being that of the EU index of Kabat. The C-terminal lysine (residue K447) of the Fc region may be removed, for example, during production or purification of an Fc fusion protein.

As used herein, the percent identity between the two sequences is a function of the number of identical positions shared by the sequences (i. e., % identity = number of identical positions/total number of positions x 100), taking into account the number of gaps, and the length of each gap, which need to be introduced for optimal alignment of the two sequences. The comparison of sequences and determination of percent identity between two sequences can be accomplished using a mathematical algorithm, as described below.

The percent identity between two amino acid sequences can be determined using the Needleman and Wunsch algorithm (NEEDLEMAN, and Wunsch).

The percent identity between two nucleotide or amino acid sequences may also be determined using for example algorithms such as EMBOSS Needle (pair wise alignment; available at www.ebi.ac.uk, Rice et al 2000 Trends Genet 16 :276-277). For example, EMBOSS Needle may be used with a BLOSUM62 matrix, a “gap open penalty” of 10, a “gap extend penalty” of 0.5, a false “end gap penalty”, an “end gap open penalty” of 10 and an “end gap extend penalty” of 0.5. In general, the “percent identity” is a function of the number of matching positions divided by the number of positions compared and multiplied by 100. For instance, if 6 out of 10 sequence positions are identical between the two compared sequences after alignment, then the identity is 60%. The % identity is typically determined over the whole length of the query sequence on which the analysis is performed. Two molecules having the same primary amino acid sequence or nucleic acid sequence are identical irrespective of any chemical and/or biological modification.

As used herein, the term “subject” includes any human or nonhuman animal. The term “nonhuman animal” preferably includes mammals, such as nonhuman primates, sheep, dogs, cats, horses, etc.

### Rspo1 Protein

The present disclosure relates to certain Rspo1 proteins or their analogues, and their use as a medicament, in particular in the treatment of diabetes in a subject in need thereof, or for inducing in vivo or in vitro the production of pancreatic beta cells, preferably human beta cells, of islets of Langerhans.

As used herein, the term « Rspo1 protein » refers to native R-spondin 1 proteins as encoded by corresponding Rspo1 gene, or any of their functional equivalents.

As used herein, the term « analogues » refers to non-protein compounds which have the same properties or substantially the same properties as R-spondin 1 protein, in particular with respect to at least one or more of the desired properties described in the next Section. Such analogues include small molecules or synthetic organic molecules of up to 2000Da, preferably up to 800Da or less, and peptidomimetics, aptamers and structural or functional mimetics of R-spondin1 protein. Analogues further include antibodies having binding specificity to LGR4, and which have the same properties or substantially the same properties as R-spondin1 protein, hereafter referred as « agonist antibodies ».

As used herein, the term « aptamer » refers to strand of oligonucleotides (DNA or RNA) that can adopt highly specific three-dimensional conformations.

The term “antibody” as used herein refers to immunoglobulin molecules and immunologically active portions of immunoglobulin molecules, i.e., molecules that contain an antigen binding site that immunospecifically binds an antigen. As such, the term “antibody” encompasses not only whole antibody molecules, but also antibody fragments as well as variants (including derivatives) of antibodies and antibody fragments.

In natural antibodies, two heavy chains are linked to each other by disulfide bonds and each heavy chain is linked to a light chain by a disulfide bond. There are two types of light chain, lambda (λ) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) can participate to the antibody binding site or influence the overall domain structure and hence the combining site. Complementarity Determining Regions or CDRs refer to amino acid sequences, which together define the binding affinity and specificity of the natural Fv region of a native immunoglobulin binding site. The light and heavy chains of an immunoglobulin each have three CDRs, designated L-CDR1, L-CDR2, L- CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. According the variable regions of the light and heavy chains typically comprise 4 framework regions and 3 CDRs of the following sequence: FR1-CDR1-FR2-CDR2-FR3-CDR3-FR4.

Agonist antibodies may thus be screened by the skilled person among anti-LGR4 antibodies obtained by conventional techniques in the art, such as hybridoma technology and/or phage display technologies, and further by selecting the anti-LGR4 antibodies which exhibit at least one or more of the desired properties described in the next Section, and using the functional assays further described in the Examples.

The term « Rspo1 protein » includes in particular any protein comprising a functional fragment of a native R-spondin 1 protein, a functional variant of a native R-spondin-1 protein, or a recombinant protein, in particular a fusion protein comprising such fragments or functional variants of a native R-spondin1 proteins, all generally referred as « functional equivalents ».

Native R-spondin 1 proteins typically include, from their N-terminal end to C-terminal end, a signal peptide (SP), two cystein-rich furin-like domains (FU1 and FU2), a thrombospondin (TSP1) motif (TSP) and a basic amino acid rich (BR) domain. FIG. 13 provides a schematic view of the different domains for human Rspondin-1. R-spondin 1 proteins are known to bind to LGR4 receptor.

Examples of Furin-like 1 domain (FU1) of human R-spondin 1 include any of SEQ ID NOs : 13-15.

Examples of Furin-like 2 domain (FU2) of human R-spondin 1 include any of SEQ ID NOs : 16-18.

In a specific embodiment, an Rspo1 protein is a protein comprising a human R-spondin 1 polypeptide, preferably of any one of SEQ ID NOs: 2-4, or a functional fragment thereof.

Another example of an Rspo1 protein is a protein comprising the murine R-spondin1 polypeptide of SEQ ID NO: 6 or a functional fragment thereof.

Examples of R-spondin 1 polypeptides or their functional fragments for use in the Rspo1 protein according to the present disclosure are described in the table 1 below:

TABLE 1

| Example | Amino Acid SEQ ID | Nucleotide SEQ ID | Brief Description |
|---|---|---|---|
| #1 | SEQ ID NO:1 | – | Human Rspo1 FU1/2 domains (region from positions 34-143) |
| #2 | SEQ ID NO:2 | – | Human Rspo1 FU1/2 and TSP1 domains (region from positions 34-207) |
| #3 | SEQ ID NO:3 | – | Human Rspo1 full-length (without SP) |
| #4 | SEQ ID NO:4 | SEQ ID NO:5 | Full-length of Human Rspo1 isoform 1 encoding gene (NP_001033722.1) |
| #5 | SEQ ID NO:8 | – | Human Rspo1 FU2 and TSP1 domains (region from positions 95-207) |
| #6 | SEQ ID NO:9 | – | Human Rspo1 FU2 and TSP1 and BR domains (region from positions 95-263) |
| #7 | SEQ ID NO:10 | – | Human Rspo1 FU1+FU2 (region from positions 34-135) |
| #8 | SEQ ID NO:11 | – | Human Rspo1 FU1+FU2 (region from positions 39-132) |
| #9 | SEQ ID NO:12 | – | Human Rspo1 FU1+FU2 (region from positions 34-143) |
| #10 | SEQ ID NO:13 | – | Human Rspo1 FU1 (region from positions 34-95) |
| #11 | SEQ ID NO:14 | – | Human Rspo1 FU1 (region from positions 34-85) |
| #12 | SEQ ID NO:15 | – | Human Rspo1 FU1 (region from positions 39-86) |
| #13 | SEQ ID NO:16 | – | Human Rspo1 FU2 (region from positions 95-143) |
| #14 | SEQ ID NO:17 | – | Human Rspo1 FU2 (region from positions 92-132) |
| #15 | SEQ ID NO:18 | – | Human Rspo1 FU2 (region from positions 91-135) |
| #16 | SEQ ID NO:19 | – | Human Rspo1 FU1 + FU2 + TSP (region from positions 34-206) |
| #17 | SEQ ID NO:20 | – | Human Rspo1 FU1 + FU2 + TSP (region from positions 39-206) |
| #18 | SEQ ID NO:21 | – | Human Rspo1 FU1 + FU2 + TSP (region from positions 39-207) |
| #19 | SEQ ID NO:22 | – | Human Rspo1 FU1 + FU2 + TSP + BR (region from positions 34-249) |
| #20 | SEQ ID NO:23 | – | Human Rspo1 FU1 + FU2 + TSP + BR (region from positions 39-263) |
| #21 | SEQ ID NO:24 | – | Human Rspo1 FU1 + FU2 + TSP + BR (region from positions 39-249) |

The following R-spondin 1 proteins or their functional fragments which are commercially available may also be used according to the present disclosure:
Full length mouse Rspo1-recombinant protein His-tag (SinoBiological Ref. 50316-M08S);
Short length mouse Rspo1-recombinant protein (aa 21-135) (CliniScience Ref. LS-G16201-100);
Human Rspo1-recombinant protein produced in CHO cells (Peprotech Ref. 120-38);
Human Rspo1-recombinant protein His-tag produced in E. coli (Creative Biomart Ref. RSP01-1942H) ;
Human Rspo1-recombinant protein Fc-tagged produced in HEK293 cells: (Creative Biomart Ref. RSP01-053H).

In more specific embodiment, said Rspo1 protein is an isolated recombinant protein comprising any one of the polypeptides of SEQ ID NOs :1-4 and SEQ ID NOs :8-24. In more specific embodiments said recombinant protein of the present disclosure is a fusion protein, for example an Fc fusion protein, typically comprising any one of the polypeptides SEQ ID NOs : 1-4 and SEQ ID NOs :8-24.

### Functional Equivalents of R-spondin 1

Additional functional equivalents of R-spondin 1 proteins with similar advantageous properties of native R-spondin 1 proteins can be further identified by screening candidate molecules and testing whether such candidate molecules have maintained the desired functional properties of the reference native R-spondin 1 protein, typically, of human Rspondin1 protein of SEQ ID NO :3 or 4.

In one embodiment, a functional equivalent of R-spondin 1 binds to LGR4 receptor.

For example, said functional equivalent of R-spondin 1 binds to LGR4 receptor with at least the same affinity as the corresponding native R-spondin 1, typically, human R-spondin 1 of SEQ ID NO:3 or 4, for example as determined by SPR assay.

In another embodiment, a functional equivalent of R-spondin 1 inhibits the binding of a native R-spondin 1, e.g human R-spondin 1, to LGR4 receptor, as determined by a competitive binding assay,

In specific embodiments, a functional equivalent of R-spondin 1 exhibits at least 50%, 60%, 70%, 80%, 90% 100% or more, of one or more the following activities relative to the corresponding native Rspondin-1, preferably to human native R-spondin 1:
(i) Binding affinity to LGR4 receptor, for example as determined by SPR assay;
(ii) Induction of the proliferation of functional beta cells, for example as determined in an in vitro beta cell proliferation assay;
(iii) Induction of the proliferation of functional beta cells, for example as determined in an in vivo beta cell proliferation assay;
(iv) Increase of glucose-stimulated insulin secretion (GSIS), for example as determined in an in vitro beta cell proliferation assay; or,
(v) Increase of glucose-stimulated insulin secretion (GSIS), for example as determined in an in vivo beta cell proliferation assay.

Further details of the assays and conditions for use in determining the activities are disclosed in the experimental part below.

In various embodiments, the functional equivalent is a recombinant protein which exhibit one, two, three, four, five or all of the desired activities discussed above. In specific embodiments, a functional equivalent is a recombinant protein which exhibit at least the desired activities (ii) to (v) as discussed above.

In specific embodiments, said functional equivalent is a recombinat protein exhibiting at least 50%, 60%, 70%, 80%, 90%, and more preferably 100% or more of the above desired activities relative to the corresponding native human R-spondin 1 of SEQ ID NO :3.

### Functional Fragments

In a specific embodiment, a functional equivalent of R-spondin 1 is a protein comprising a fragment of a native R-spondin 1 polypeptide.

In specific embodiments, a « fragment of Rspondin 1 polypeptide » refers to a polypeptide having at least 40, 50, 60, 70, 80, 90, 100, 110, 120, 130, 140, 150, 160, 170, 180, 190, 200, 210, consecutive amino acid residues of any of the polypeptides of SEQ ID NOs :1-4 and SEQ ID NOs :8-24.

A fragment of R-spondin 1 is by definition at least one amino acid shorter than full length wild-type R-spondin 1. In specific embodiments, said fragment of R-spondin 1 lacks the Thrombospondin-1 Domains (TSP1 et TSP2) and/or the Basic amino-acid Rich Domain (BR).

In specific embodiments, said fragment of R-spondin 1 comprises at least 40-52 consecutive amino acids of the FU2 domain (e.g. from residue 91 to residue 143 of human R-spondin 1 of SEQ ID NO :4), and/or at least 40-61 consecutive amino acids of the FU1 domain of R-spondin 1 protein (e.g. from residues 34 to residue 95 of human R-spondin 1 of SEQ ID NO :4).

In more specific embodiments, a « fragment of R-spondin 1 » refers to a polypeptide having
(i) any one of SEQ ID NOs: 1-4 and SEQ ID NOs:8-24, or
(ii) a combination of fragments of R-spondin 1 polypeptide of SEQ ID NO:1, typically including the functional domain FU1 and the functional domain FU2, and, optionally the functional domain TSP.

Hence, in particular embodiments, a functional equivalent of R-spondin 1 is a protein comprising
(i) any one of SEQ ID NOs: 1-4 and SEQ ID NOs:8-24, or
(ii) a combination of fragments of R-spondin-1 polypeptide of SEQ ID NO:1, typically including the functional domain FU1 and the functional domain FU2, and, optionally the functional domain TSP;
and wherein said protein exhibits at least 50%, 60%, 70%, 80%, 90% 100% or more of the following activities relative to the corresponding native R-spondin 1:
(i) Binding affinity to LGR4 receptor, for example as determined by SPR assay;
(ii) Induction of the proliferation of functional beta cells, for example as determined in an in vitro beta cell proliferation assay;
(iii) Induction of the proliferation of functional beta cells, for example as determined in an in vivo beta cell proliferation assay;
(iv) Increase of glucose-stimulated insulin secretion (GSIS), for example as determined in an in vitro beta cell proliferation assay; or,
(v) Increase of glucose-stimulated insulin secretion (GSIS), for example as determined in an in vivo beta cell proliferation assay.

### Functional Mutant Variants

In specific embodiments, said functional equivalent is a protein comprising a functional variant of the functional domains FU1 and/or FU2 of R-spondin 1, typically of human R-spondin 1.

In specific embodiments, said « functional variant » comprises or essentially consists of a polypeptide having at least 50%, 60%, 70%, 80%, 90% or at least 95% identity to a parent (native) R-spondin 1 protein or to a functional fragment of a parent (native) R-spondin 1. In specific embodiments, said functional variant has at least 50%, 60%, 70%, 80%, 90% or at least 95% identity to one of the parent polypeptide of any one of SEQ ID NOs: 1-4 and SEQ ID NO :8-24.

The functional variants may be a mutant variant obtained typically by amino acid substitution, deletion or insertion as compared to the corresponding native polypeptide or their functional fragments. In certain embodiments, a functional variant may have a combination of amino acid deletions, insertions or substitutions throughout its sequence, as compared to the parent polypeptide. In a particular embodiment, said functional variant differ from the corresponding native R-spondin 1 sequence or its functional fragment, through only amino acid substitutions, with natural or non-natural amino acids, preferably only 1, 2, 3, 4, 5, 6, 7, 8, 9 or 10 amino acid substitutions with natural amino acids, in particular as compared to one of the native R-spondin 1 polypeptides of SEQ ID NOs :1-4 and SEQ ID NOs :8-24. In a specific embodiment, a functional variant is a mutant variant having 1, 2 or 3 amino acid substitutions as compared to a human R-spondin 1 of SEQ ID NO:4.

In other embodiments, said functional mutant variant is a polypeptide having at least 50%, 60%, 70%, 80%, 90% or at least 95% identity to a parent (native) R-spondin 1 protein or its functional fragment, for example to a polypeptide of any of SEQ ID NO :1-4 and SEQ ID NO :8-24, and wherein said polypeptide comprises a FU1 domain which is 100% identical to the FU1 domain of the corresponding native R-spondin 1 protein, typically human R-spondin 1 protein.

In other embodiments, said functional mutant variant is a polypeptide having at least 50%, 60%, 70%, 80%, 90% or at least 95% identity to a parent (native) R-spondin 1 protein or its functional fragment, for example to a polypeptide of any of SEQ ID NOs :1-4 and SEQ ID NO :8-24, and wherein said polypeptide comprises a FU2 domain which is 100% identical to the FU2 domain of the corresponding native R-spondin 1 protein, typically human R-spondin 1 protein.

In other embodiments, said functional mutant variant is a polypeptide having at least 50%, 60%, 70%, 80%, 90% or at least 95% identity to a parent (native) R-spondin 1 protein, for example to a polypeptide of any of SEQ ID NOs :1-4 and SEQ ID NO :8-24, and wherein said polypeptide comprises FU1 and FU2 domains which are 100% identical to the corresponding FU1 and FU2 domains respectively of the native R-spondin 1 protein, typically human R-spondin 1 protein.

In more specific embodiments, the amino acid sequence of said functional variant may differ from the native R-spondin 1 sequence or its functional fragment through mostly conservative amino acid substitutions ; for instance at least 10, such as at least 9, 8, 7, 6, 5, 4, 3, 2 or 1 of the substitutions in the variant are conservative amino acid residue replacements.

In the context of the present disclosure, conservative substitutions may be defined by substitutions within the classes of amino acids reflected as follows:
Aliphatic residues I, L, V, and M
Cycloalkenyl-associated residues F, H, W, and Y
Hydrophobic residues A, C, F, G, H, I, L, M, R, T, V, W, and Y
Negatively charged residues D and E
Polar residues C, D, E, H, K, N, Q, R, S, and T
Positively charged residues H, K, and R
Small residues A, C, D, G, N, P, S, T, and V
Very small residues A, G, and S
Residues involved in turn A, C, D, E, G, H, K, N, Q, R, S, P, and formation T
Flexible residues Q, T, K, S, G, P, D, E, and R

More conservative substitutions groupings include: valine-leucine-isoleucine, phenylalanine-tyrosine, lysine-arginine, alanine-valine, and asparagine-glutamine. Conservation in terms of hydropathic/hydrophilic properties and residue weight/size also may be substantially retained in a variant mutant polypeptide as compared to a parent polypeptide of any one of SEQ ID NOs :1-4 or SEQ ID NOs 8-24.

In specific embodiments, a functional variant comprises a polypeptide which is identical to any one of SEQ ID NOs :1-4 or SEQ ID NOs : 8-24, except for 1, 2 or 3 amino acid residues which have been replaced by another natural amino acid, preferably by conservative amino acid substitutions as defined above.

Xu et al (Journal of Biological Chemistry, 2015, Vol 290, No4, pp 2455-2465) have described the crystal structure of LGR4-Rspo1 complex. They report in particular that the two central tandem FU1/FU2 domains of human Rspo1 are required for binding to LGR receptors, in particular residues 34-135. More specifically, they report that the linking loops of the β4-β3, β5-β6 and β8-β7 hairpins of human Rspo1, located on the same side of Rspo1 are responsible for binding to LGR4.

Hence, in other specific embodiments that may be combined with the previous embodiments, a functional mutant variant of human R-spondin 1 comprises at least the following amino acid residues of human R-spondin 1 protein : Asp-85, Arg-87, Phe-107, Asn-109, Phe-110 and Lys-122. In other specific embodiments, that may be combined with the previous embodiments, the conserved cysteines at amino acid residues 53, 56, 94, 97, 102, 106, 111, 114, 125 and 129 may also not be mutated (see also FIG. 3 of Xu et al 2015).

In addition, the person skilled in the art will appreciate that the conserved residues among various species may be important to maintain the proper structure and therefore may refrain from mutating such amino acid positions. Alternatively, at many sites, one or two or more amino acids positions show conservative variations among species variants, and/or among other members of Rspo family, such as Rspo2, Rpo3 and Rspo4 : One of skill in the art would understand that some of such conservative substitutions may likely not adversely affect the function of Rspo1 and may therefore by mutated as compare to native R-spondin 1 with such conservative variations.

In particular, in other specific embodiments, a functional variant therefore comprises a polypeptide sequence almost identical to human R-spondin 1 except that it includes one or more of the following amino acid susbstitutions or deletions:

K115Q, S134T, G138S, S143G, Q163R, Q164K, R170K, V184G, A188T, A189T, R198K, V204T, N226H, L227P, E231N, A235P, A237S, G238N, R242H, ΔQ248, Q251P, V254T, A260V, A263T.

These amino acid substitutions correspond to the amino acid substitutions from human Rspondin1 to mouse R-spondin 1 when the two sequences are aligned as shown in FIG. 12.

Further variations may be tolerated at other sites within R-spondin 1 without effect on function. For example, the skilled person may also identify other possible amino acid substitutions or insertions for identifying functional variants by comparing the alignment of human Rspondin1 and other mammal Rspondin1 proteins, such as primates, rats, canine, feline etc.

Any functional variants of R-spondin 1 may also be screened for their capacity to maintain the advantageous desired properites of the native R-spondin 1 polypeptide, as described above, and using the functional assays as described in the experimental part below.

### Recombinant Protein of the Disclosure

In particular embodiments, said Rspo1 protein of the disclosure is a soluble and/or a recombinant protein.

In more specific embodiments, said recombinant Rspo1 protein is a fusion protein, and more specifically an Fc fusion protein.

A variety of polypeptides other than R-spondin 1 polypeptides can be fused to a R-spondin 1 polypeptide or its functional equivalents as described above (in particular fragments or mutant variants), for a variety of purposes such as, for example, to increase in vivo half life of the protein, to facilitate identification, isolation and/or purification of the protein, to increase the activity of the protein, and to promote oligomerization of the protein.

Many polypeptides can facilitate identification and/or purification of a recombinant fusion protein of which they are a part. Examples include polyarginine, polyhistidine. Polypeptides comprising polyarginine allow effective purification by ion exchange chromatography.

In a specific embodiment, a polypeptide that comprises an Fc region of an antibody, optionally an IgG antibody, or a substantially similar protein, can be fused to a R spondin-1 polypeptide, directly, or optionally via a peptidic linker, thereby forming an Fc fusion protein of the present disclosure.

Another modification of the antibodies that is contemplated by the present disclosure is a conjugate or a protein fusion of at least the R spondin-1 polypeptides (or functional fragment or variant thereof) to a serum protein, such as human serum albumin or a fragment thereof to increase half-life of the resulting molecule. Such approach is for example described in Ballance et al. EP 0 322 094.

Another possibility is a fusion protein of the disclosure including proteins capable of binding to serum proteins, such as binding to human serum albumin (i.e. anti-HSA fusion protein) to increase half life of the resulting molecule, including for example anti-HSA binding moieties derived from Fab or nanobody that binds to HSA or any other domain type structures such as darpin, nanofitin, fynomer and the like. Such approach is for example described in Nygren et al., EP 0 486 525.

A recombinant fusion protein of the disclosure can comprise a polypeptide comprising a leucine zipper or other multimerization motifs. Among known leucine zipper sequences are sequences that promote dimerization and sequences that promote trimerization. See e.g. Landschulz et al. (1988), Science 240: 1759-64). Leucine zippers comprise a repetitive heptad repeat, often with four or five leucine residues interspersed with other amino acids. Use and preparation of leucine zippers are well known in the art.

A fusion protein may also comprise one or more peptide linkers. Generally, a peptide linker is a stretch of amino acids that serves to link plural polypeptides to form multimers and provides the flexibility or rigidity required for the desired function of the linked portions of the protein. Typically, a peptide linker is between about 1 and 30 amino acids in length. Examples of peptide linkers include, but are not limited to, -Gly-Gly-, GGGGS (SEQ ID NO :25), (GGGGS)n (wherein n is between 1-8, typically 4). Linking moieties are described, for example, in Huston, J. S., et al., Proc. Natl. Acad. Sci. 85: 5879-83 (1988), Whitlow, M., et al., Protein Engineering 6: 989-95 (1993), Newton, D. L., et al., Biochemistry 35: 545-53 (1996), and U.S. Pat. Nos. 4,751,180 and 4,935,233.

A recombinant Rspo1 protein according to the present disclosure can comprise a R-spondin 1 protein or its functional equivalent, that lacks its normal signal sequence and has instead a different signal sequence replacing it. The choice of a signal sequence depends on the type of host cells in which the recombinant protein is to be produced, and a different signal sequence can replace the native signal sequence.

Another modification of the R-spondin 1 protein or related recombinant Rspo1 proteins herein that is contemplated by the present disclosure is pegylation or hesylation or related technologies such as PASylation.

More generally, the Rspo1 protein may be conjugated with biodegradable bulking agents, including natural and semi-synthetic polysaccharides, ncluding O- and N-linked oligosaccharides, dextran, hydroxyethylstarch (HES), polysialic acid and hyaluronic acid, as well as unstructured protein polymers such as homo-amino acid polymers, elastin-like polypeptides, XTEN and PAS

A Rspo1 protein of the disclosure can be pegylated to, for example, increase the biological (e.g., serum) half-life of the antibody. To pegylate an Rspo1 protein is reacted with polyethylene glycol (PEG), such as a reactive ester or aldehyde derivative of PEG, under conditions in which one or more PEG groups become attached to the Rspo1 protein. The pegylation can be carried out by an acylation reaction or an alkylation reaction with a reactive PEG molecule (or an analogous reactive water-soluble polymer).

As used herein, the term “polyethylene glycol” is intended to encompass any of the forms of PEG that have been used to derivatize other proteins, such as mono (C1-C10) alkoxy-or aryloxy-polyethylene glycol or polyethylene glycol-maleimide. Methods for pegylating proteins are known in the art and can be applied to the proteins of the disclosure. See for example, Jevsevar et al 2010 Biotechnol J. 5(1) : 113-28, or Turecek et al 2016 J Pharm Sci 2016 105(2) : 460-375. Hence, in specific embodiments, the Rspo1 protein of the disclosure is pegylated.

Another modification of the Rspo1 protein or related recombinant proteins that is contemplated by the present disclosure is PASylation. See for example: Protein Engineering, Design & Selection vol. 26 no. 8 pp. 489-501, 2013. Hence, in specific embodiments, the Rspo1 protein of the disclosure is PASylated.

Xten technology is for example described in are reviewed for example in Nature Biotechnology volume 27 number 12 2009: 1186-1192.

### Nucleic Acid Molecules Encoding the Proteins of the Disclosure

Also disclosed herein are the nucleic acid molecules that encode the Rspo1 proteins of the disclosure.

Examples of nucleotide sequences are those encoding the amino acid sequences of any one of examples #1-#21, as defined in the above Table 1, in particular encoding any one of SEQ ID NO :1-4 and SEQ ID NO8-24, the nucleic acid sequences being easily derived from the Table 1, and using the genetic code and, optionally taking into account the codon bias depending on the host cell species.

The present disclosure also pertains to nucleic acid molecules that derive from the latter sequences having been optimized for protein expression in mammalian cells, for example, mammalian Chinese Hamster Ovary (CHO) cell lines.

The nucleic acids may be present in whole cells, in a cell lysate, or may be nucleic acids in a partially purified or substantially pure form. A nucleic acid is “isolated” or “rendered substantially pure” when purified away from other cellular components or other contaminants, e.g., other cellular nucleic acids or proteins, by standard techniques, including alkaline/SDS treatment, CsCI banding, column chromatography, agarose gel electrophoresis and others well known in the art. A nucleic acid of the disclosure can be, for example, DNA or RNA and may or may not contain intronic sequences. In an embodiment, the nucleic acid may be present in a vector such as a phage display vector, or in a recombinant plasmid vector.

Nucleic acids of the disclosure can be obtained using standard molecular biology techniques. Once DNA fragments encoding, for example, Rspo1 encoding fragments are obtained, these DNA fragments can be further manipulated by standard recombinant DNA techniques. In these manipulations, a Rspot-encoding DNA fragment may be operatively linked to another DNA molecule, or to a fragment encoding another protein, such as an antibody constant region (Fc region) or a flexible linker. Examples of nucleotide sequences further include nucleotide sequences encoding a recombinant fusion protein, in particular an Fc fusion protein comprising coding sequences of any one of the amino acid sequences SEQ ID Nos 1-4 and 8-24 operatively linked with a coding sequence of an Fc region.

The term “operatively linked”, as used in this context, is intended to mean that the two DNA fragments are joined in a functional manner, for example, such that the amino acid sequences encoded by the two DNA fragments remain in-frame, or such that the protein is expressed under control of a desired promoter.

### Generation of Transfectomas Producing Rspo1 Proteins of the Disclosure

The Rspo1 proteins of the present disclosure can be produced in a host cell transfectoma using, for example, a combination of recombinant DNA techniques and gene transfection methods as is well known in the art.

For example, to express the Rspo1 proteins, or corresponding fragments thereof, DNAs encoding partial or full-length recombinant proteins can be obtained by standard molecular biology or biochemistry techniques (e.g., DNA chemical synthesis, PCR amplification or cDNA cloning) and the DNAs can be inserted into expression vectors such that the genes are operatively linked to transcriptional and translational control sequences.

In this context, the term “operatively linked” is intended to mean that an antibody gene is ligated into a vector such that transcriptional and translational control sequences within the vector serve their intended function of regulating the transcription and translation of the recombinant protein. The expression vector and expression control sequences are chosen to be compatible with the expression host cell used. The protein encoding genes are inserted into the expression vector by standard methods (e.g., ligation of complementary restriction sites on the antibody gene fragment and vector, or blunt end ligation if no restriction sites are present).

The recombinant expression vector can encode a signal peptide that facilitates secretion of the recombinant protein from a host cell. The Rspo1 encoding gene can be cloned into the vector such that the signal peptide is linked in frame to the amino terminus of the recombinant protein. The signal peptide can be the native signal peptide of Rspo1 or a heterologous signal peptide (i.e., a signal peptide from a non-Rspo1 protein).

In addition to the Rspo1 protein encoding genes, the recombinant expression vectors disclosed herein carry regulatory sequences that control the expression of the recombinant protein in a host cell. The term “regulatory sequence” is intended to include promoters, enhancers and other expression control elements (e.g., polyadenylation signals) that control the transcription or translation of the protein encoding genes. It will be appreciated by those skilled in the art that the design of the expression vector, including the selection of regulatory sequences, may depend on such factors as the choice of the host cell to be transformed, the level of expression of protein desired, etc. Regulatory sequences for mammalian host cell expression include viral elements that direct high levels of protein expression in mammalian cells, such as promoters and/or enhancers derived from cytomegalovirus (CMV), Simian Virus 40 (SV40), adenovirus (e.g., the adenovirus major late promoter (AdMLP)), and polyoma. Alternatively, nonviral regulatory sequences may be used, such as the ubiquitin promoter or P-globin promoter. Still further, regulatory elements composed of sequences from different sources, such as the SRa promoter system, which contains sequences from the SV40 early promoter and the long terminal repeat of human T cell leukemia virus type 1.

In addition to the Rspo1 protein encoding genes and regulatory sequences, the recombinant expression vectors of the present disclosure may carry additional sequences, such as sequences that regulate replication of the vector in host cells (e.g., origins of replication) and selectable marker genes. The selectable marker gene facilitates selection of host cells into which the vector has been introduced (see, e.g., U.S. Patent Nos. 4,399,216, 4,634,665 and 5,179,017, all by Axel et al.). For example, typically the selectable marker gene confers resistance to drugs, such as G418, hygromycin or methotrexate, on a host cell into which the vector has been introduced. Selectable marker genes include the dihydrofolate reductase (DHFR) gene (for use in dhfr- host cells with methotrexate selection/amplification) and the neo gene (for G418 selection).

For expression of the Rspo1 proteins, the expression vector(s) encoding the recombinant protein is transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. It is theoretically possible to express the proteins of the present disclosure in either prokaryotic or eukaryotic host cells. Expression of proteins in eukaryotic cells, for example mammalian host cells, yeast or filamentous fungi, is discussed because such eukaryotic cells, and in particular mammalian cells, are more likely than prokaryotic cells to assemble and secrete a properly folded and functional Rspo1 protein.

In one specific embodiment, a cloning or expression vector according to the disclosure comprises one of the coding sequences of the Rspo1 proteins of any one of SEQ ID NOs :1-4, and SEQ ID NOs :8-24, operatively linked to suitable promoter sequences.

Mammalian host cells for expressing the recombinant proteins of the disclosure include Chinese Hamster Ovary (CHO cells), including dhfr- CHO cells (described in Urlaub and Chasin, 1980) used with a DHFR selectable marker(as described in Kaufman and Sharp, 1982), CHOK1 dhfr+ cell lines, NSO myeloma cells, COS cells and SP2 cells. When recombinant expression vectors encoding antibody genes are introduced into mammalian host cells, the recombinant proteins are produced by culturing the host cells for a period of time sufficient for expression of the recombinant proteins in the host cells and, optionally, secretion of the proteins into the culture medium in which the host cells are grown.

The recombinant proteins of the disclosure can be recovered and purified for example from the culture medium after their secretion using standard protein purification methods.

In one specific embodiment, the host cell of the disclosure is a host cell transfected with an expression vector having the coding sequences suitable for the expression of a Rspo1 protein comprising any one of SEQ ID NOs :1-4 and SEQ ID NOs :8-24, respectively, operatively linked to suitable promoter sequences.

For example, the present disclosure relates to a host cell comprising at least the nucleic acid of SEQ ID NO:5 encoding human R-spondin 1 protein.

The latter host cells may then be further cultured under suitable conditions for the expression and production of a recombinant protein of the disclosure.

### Pharmaceutical Compositions

In another aspect, the present disclosure provides a composition, e.g., a pharmaceutical composition, containing one or a combination of Rspo1 protein, or an analogue thereof, as disclosed herein. Such compositions may include one or a combination of (e.g., two or more different) Rspo1 proteins, as described above.

For example, said pharmaceutical composition comprises a recombinant protein comprising a polypeptide of any one of SEQ ID NOs : 1-4 and SEQ ID NOs :8-24, or a functional variant thereof, formulated together with a pharmaceutically acceptable carrier.

Pharmaceutical compositions disclosed herein can also be administered in combination therapy, i.e., combined with other agents. For example, the combination therapy can include an Rspo1 protein of the present disclosure, for example a recombinant protein comprising a polypeptide of any one of SEQ ID NOs :1-4 and SEQ ID NOs :8-24 or a functional variant thereof, combined with at least one anti-inflammatory, or another anti-diabetic agent. Examples of therapeutic agents that can be used in combination therapy are described in greater detail below in the section on uses of the Rspo1 proteins of the disclosure.

As used herein, “pharmaceutically acceptable carrier” includes any and all solvents, dispersion media, coatings, antibacterial and antifungal agents, isotonic and absorption delaying agents, and the like that are physiologically compatible. The carrier should be suitable for a parenteral, intranasal, intravenous, intramuscular, subcutaneous or intraocular administration (e.g., by injection or infusion).

In one embodiment, the carrier should be suitable for subcutaneous route or intravenous injection. Depending on the route of administration, the active compound, i.e., the Rspo1 protein, may be coated in a material to protect the compound from the action of acids and other natural conditions that may inactivate the compound.

Sterile phosphate-buffered saline is one example of a pharmaceutically acceptable carrier. Other suitable carriers are well-known to those in the art. (Remington and Gennaro, 1995). Formulations may further include one or more excipients, preservatives, solubilizers, buffering agents, albumin to prevent protein loss on vial surfaces, etc.

The form of the pharmaceutical compositions, the route of administration, the dosage and the regimen naturally depend upon the condition to be treated, the severity of the illness, the age, weight, and sex of the patient, etc.

The pharmaceutical compositions of the disclosure can be formulated for oral, intranasal, sublingual, subcutaneous, intramuscular, intravenous, transdermal, parenteral, toptical, intraocular, or rectal administration and the like. The Rspo1 protein as an active principle, alone or in combination with another active principle, can be administered in a unit administration form, as a mixture with conventional pharmaceutical supports, to animals and human beings.

Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

Preferably, the pharmaceutical compositions contain vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the Rspo1 proteins may be dissolved or dispersed in a pharmaceutically acceptable carrier or aqueous medium.

The pharmaceutical forms suitable for injectable use may include sterile aqueous solutions or dispersions; formulations including sesame oil, peanut oil or aqueous propylene glycol; and sterile powders or lyophilisates for the extemporaneous preparation of sterile injectable solutions or dispersions. In all cases, the form must be sterile and must be fluid to the extent that easy syringeability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An Rspo1 protein of the disclosure can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compounds, i.e. the Rspo1 proteins, in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington’s Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The Rspo1 proteins of the disclosure or their analogue may be formulated within a therapeutic mixture to comprise about 0.01 mg -1000 mg/kg or 1 mg - 100 mg/kg. Multiple doses can also be administered.

Suitable formulation for solution for infusion or subcutaneous injection of the recombinant proteins have been described in the art and for example are reviewed in Advances in Protein Chemistry and Structural Biology Volume 112, 2018, Pages 1-59 Therapeutic Proteins and Peptides Chapter One - Rational Design of Liquid Formulations of Proteins: Mark C.Manning, Jun Liu, Tiansheng Li, Ryan E.Holcomb.

### Uses and Methods of the Proteins of the Disclosure

The Rspo1 proteins of the present disclosure have in vitro and in vivo utilities. For example, these molecules can be administered to cells in culture, e.g. in vitro, ex vivo or in vivo, or in a subject, e.g., in vivo, to treat, or prevent a variety of disorders.

As used herein, the term “treat” “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease.

In particular, with reference to the treatment of a diabetes, more specificalyl diabete type 1, the term “treatment” may refer to the inhibition of the loss of pancreatic beta cells, and/or the increase of the mass of pancreatic beta cells, in particular functional insulin secreting beta-cells in said subject, and/or improvement of glycemia control, in particular in patients having loss of pancreatic beta cells and/or islets of Langerhans due to a disease, for example diabete type 1.

The Rspo1 proteins of the disclosure or their analogue can induce the proliferation of pancreatic beta cells in vivo and reconstitute functional insulin-secreting islets of Langerhans, and thereby may be used to treat diabetic patients, or patients in need of functional insulin-secreting beta cells, or patients with disorders associated with hyperglycemia, or patients with deficient glucose stimulated insulin secretion.

As used herein, the terms “diabetes” generally refer to any conditions or disorders resulting in insulin shortage or resistance to its action

Examples of diabetes include, but are not limited to, type 1, type 2, gestional, and Latent autoimmune diabetes in adults (LADA).

Accordingly, the disclosure relates to a method for treating one of the disorders disclosed above, in a subject in need thereof, said method comprising administering to said subject a therapeutically efficient amount of an Rspo1 protein or analogue as disclosed above, typically, a recombinant protein comprising a polypeptide of any one of SEQ ID NOs:1-4 and SEQ ID NOs:8-24 or a functional variant thereof.

In certain embodiments, said subject has been selected among patient having low Rspo1 gene expression.

The Rspo1 proteins or analogue for use as disclosed above may be administered as the sole active ingredient or in conjunction with, e.g. as an adjuvant to or in combination to, other drugs e.g. cytokines, anti-viral, anti-inflammatory agents, anti-diabetic or hypoglycemiant agents, cell therapy product (e.g beta cell composition) and immune modulatory drugs, e.g. for the treatment or prevention of diseases mentioned above.

For example, the Rspo1 proteins or analogue for use as disclosed above may be used in combination with cell therapy, in particular β cell therapy.

As used herein, the term “cell therapy” refers to a therapy comprising the in vivo administration of at least a therapeutically efficient amount of a cell composition to a subject in need thereof. The cells administered to the patient may be allogenic or autologous. The term “β cell therapy” refers to a cell therapy wherein the cell composition includes, as the active principle, β cells, in particular insulin secreting beta cells. Such beta cells may be produced using the Rspo1 proteins in an in vitro method as described hereafter.

A cell therapy product refers to the cell composition which is administered to said patient for therapeutic purposes. Said cell therapy product include a therapeutically efficient dose of cells and optionally, additional excipients, adjuvants or other pharmaceutically acceptable carriers.

Suitable anti-diabetic or hypoglycemiant agents may include without limitation, angiotensin-converting enzyme inhibitors, angiotensin II receptor blockers, cholesterol lowering drugs, biguanides, metformine, thiazolidinediones, hypoglycemiant sulfamides, DPP-4 inhibitors, alpha-glucosidases inhibitors, insulin or their derivatives, including short-acting, rapid-acting or long-acting insulin, GLP1 analogues, derivatives of carbamoylmethylbenzoic acid ; typically, insulin receptors, SLGT2 inhibtiors, GABR targeting molecules, and IL2R targeting molecule.

In accordance with the foregoing the present disclosure provides in a yet further aspect:

A method as defined above comprising co-administration, e.g. concomitantly or in sequence, of a therapeutically effective amount of an Rspo1 protein of the disclosure or analogue, and at least one second drug substance, said second drug substance being cytokines, anti-viral, anti-inflammatory agents, anti-diabetic agents, cell therapy product (e.g beta cell composition), e.g. as indicated above.

In another embodiment, the Rspo1 proteins or analogue of the disclosure can be used in in vitro methods to induce the proliferation of pancreatic beta cells and/or islets of Langerhans.

Accordingly, in one aspect, the disclosure further provides methods for in vitro producing beta-cells said method comprising
(i) providing beta-cells,
(ii) culturing said beta-cells in the presence of an efficient amount of said Rspo1 protein or analogue of the present disclosure under conditions to induce the proliferation of said beta-cells.

In specific embodiments of said production method, said beta-cells are primary cells, preferably from a subject in need of beta-cells therapy or transplantation of islets of Langerhans.

In other specific embodiments, said beta-cells provided at step (i) have been obtained from iPS cells, after differentiating said iPS cells into beta-cells.

Accordingly, in a particular embodiment, the disclosure relates to in vitro method for the production of beta-cells from induced pluripotent stem cells, comprises the following:
(i) providing induced pluripotent stem cells (iPSCc),
(ii) in vitro differentiating said iPSCs to β-cells of islets of Langerhans, and
(iii) culturing said differentiated beta-cells under proliferating conditions,
wherein a sufficient amount of said Rspo1 protein or analogue is added at step (ii) and/or step (iii) for differentiating iPS cells and/or inducing the proliferation of said β-cells.

Methods for differentiating iPSCs to β-cells of islets of Langerhans are already described in the art, for example in Pagliuca, et al. Cell 159, 428-439 (2014) and Rezania et al. Nat Biotechnol. 2014 Nov;32(11):1121-33), the relevant part being incorporated within the present disclosure.

Said disclosure further includes the composition comprising said β-cells obtainable or as obtained by the above methods and their use as a cell therapy product, for example in a subject for treating diabete, preferably diabete type 1. Methods for transplanting beta-cells or islets of Langerhans to patients are for example disclosed in Shapiro, et al (2000) The New England Journal of Medicine. 343 (4): 230-238, and Shapiro et al (2017) Nature Reviews Vol 13 : 268-277.

Also within the scope of the present disclosure are kits consisting of the compositions (e.g., the Rspo1 proteins of the disclosure) disclosed herein and instructions for use. The kit can further contain a least one additional reagent, or one or more additional antibodies or proteins. Kits typically include a label indicating the intended use of the contents of the kit. The term label includes any writing, or recorded material supplied on or with the kit, or which otherwise accompanies the kit. The kit may further comprise tools for diagnosing whether a patient belongs to a group that will respond to an Rspo1 treatment, as defined above.

Another therapeutic strategy is based on the use of the Rspo1 proteins as disclosed herein as agents which expand beta cells isolated from a sample of a human subject.

The disclosure thus relates to a method for treating a subject in need thereof, comprising:
(a) isolating cells from a subject,
(b) optionally expanding and/or reprogramming said cells to induced pluripotent stem cells,
(c) differentiating said iPS cells to beta cells, and
(d) expanding in vitro said beta cells in the presence of a Rspo1 protein or analogue as disclosed herein, for example a recombinant protein comprising any one of SEQ ID NOs :1-4 and SEQ ID NOs :8-24, or a functional variant thereof, and, optionally, other cells,
(e) optionally, collecting the expanded beta cells, and/or formulating the expanded beta cells and administering a therapeutically efficient amount of said expanded beta cells to the subject.

The disclosure further relates to the use of said Rspo1 proteins disclosed herein (such as a recombinant protein comprising any one of SEQ ID NO: 1-4 and SEQ ID NO:8-24 or a functional variant thereof) as agents which in vitro expand beta cells.

The disclosure also relates to the Rspo1 proteins disclosed herein (such as a recombinant protein comprising any one of SEQ ID NO:1-4 and SEQ ID NO:8-24 or a functional variant thereof) for use in vivo as an agent for inducing the proliferation of beta-cells in human, in particular in a subject that has a loss of functional beta-cells, typically a subject suffering from diabete.

The disclosure thus relates to a method of treatment of a subject suffering from diabete, e.g. diabete type-1 or another disorder with a loss of functional beta cells, said method comprising:
(i) administering in said subject an efficient amount of an Rspo1 protein or analogue as disclosed herein, typically an Rspo1 protein, and,
(ii) administering an efficient amount of a β cell composition in said subject,
wherein said efficient amount of Rspo1 protein or analogue has the capacity to increae the proliferation of said β cell composition. Steps (i) and (ii) can be carried out simultaneously or sequentially, in particular, either step (i) or step (ii) is first administered to said subject.

The invention having been fully described is now further illustrated by the following examples, which are illustrative only and are not meant to be further limiting.

### DESCRIPTION OF THE FIGURES

FIG. 1: RT-qPCR analyses of R-spondin genes expression in WT mouse pancreata. Rspo1 is expressed in the mouse pancreas, conversely Rspo2 and Rspo4 are not detected. (n=5, age=3 months, p < 0.0001 (****), p < 0.001 (***), p < 0.01 (**), and p < 0.05.

FIG. 2: RT-qPCR analyses of Rspo1 expression in the mouse pancreas from embryonic day 15.5 (E15.5) up to 9 months of age. (n=5, p < 0.0001 (****), p < 0.001 (***), p < 0.01 (**), and p < 0.05 (*)).

FIG. 3: RNAscope of adult pancreas labeled with Rspo1 probe. The expression of both RNAs is restricted to acinar cells (dots within cells) within the exocrine compartment.

FIG. 4 : IPGTT in Rspo1K0 mice. Rspo1 loss leads to improved glucose tolerance with a significant reduction of the glycemic peak. (n=5, age=2.5 months, p < 0.0001 (****), p < 0.001 (***), p < 0.01 (**), and p < 0.05 (*))

FIG. 5 : Quantitative analysis of Rspo1K0 mice pancreata. Rspo1 deficiency does not induce any structural change of the islets of Langerhans,the total islet surface resulting unchanged upon Rspo1 loss (A). Indeed, Rspo1K0 mice do not shown any change in insulin- (B), glucagon- (C) and somatostatin-producing cell count (D). (n=5 ; age=3 months, p < 0.0001 (****), p<0.001 (***), p<0.01 (**), and p<0.05 (*))

FIG. 6: Body weight and basal glycemia of wild-type mice treated with Rspo1-recombinant protein. Rspo1-recombinant protein treatment does not induce any change of body weight and basal glycemia (n=6; age=2 months at the beginning of the treatment, p < 0.0001 (****), p < 0.001 (***), p < 0.01 (**), and p < 0.05 (*))

FIG. 7: IPGTT and insulinemia measurement upon Rspo1 treatment. Treated animals display a better glucose tolerance compared to age-matched control mice, with a strong reduction of the glycemic peak and a faster return to euglycemia (A). The enhanced glucose tolerance is caused by an increased glucose-stimulated insulin secretion upon Rspo1 administration (B) (n=6, age=3 months, p < 0.0001 (****), p < 0.001 (***), p < 0.01 (**), and p < 0.05 (*))

FIG. 8: Immunofluorescence analyses of paraffin pancreatic section upon Rspo1 administration. Mice treated with Rspo1-recombinant protein display a significant islet hypertrophy (light gray) and an increase in the number of proliferating β-cells, marked with BrdU (in white).

FIG. 9: Quantitative analyses of WT pancreata upon Rspo1-recombinant protein injections. Mice injected daily with Rspo1 showed a significantly increased β-cell proliferation (A) Consequently, islet area resulted increased in mice treated with recombinant Rspo1 compared to age-matched controls only injected with saline (B). Rspo1-recombinant protein administration significantly increases β-cell mass (C) but does not show any effect on α-cell number (D). (n=6, age=3 months, p < 0.0001 (****), p < 0.001 (***), p < 0.01 (**), and p < 0.05 (*).

FIG. 10: Rspo1 treatment induces functional beta-cell neogenesis upon beta-cell ablation. WT mice were subjected to high dose streptozotocin (STZ) treatment to ablate beta-cells and then treated with Rspo1 (or saline) once they were overtly diabetic (glycemia ≥ 300 mg/dl). While saline-treated animals developed a massive hyperglycemia, their Rspo1-treated counterparts, following a peak in glycemia, saw a progressive normalization of their blood glucose levels. Quantitative immunohistochemical analyses (percentages in colored rectangles) during the course of these experiments outlined a loss of beta-cells post-STZ. Interestingly, upon Rspo1 treatment, a progressive increase in insulin+ cell count was observed, this continued augmentation eventually resulting in the replenishment of the whole beta-cell mass.

FIG. 11: Rspo1 treatment induces human beta-cell proliferation. Human islets were cultured for 5 days in presence or not of Rspo1 and in presence of BrdU. Immunohistochemical analyses outlined very few proliferating (white dots) insulin-producing cells in controls (left). Interestingly, upon Rspo1 treatment (right), a massive increase in the number of human proliferating beta-cells was outlined, demonstrating that Rspo1 can also induce human beta-cell proliferation.

FIG. 12: Alignments between Rspondin1 human and murine sequences obtained online using Clustal Omega using the defaults settings (https://www.ebi.ac.uk/Tools/msa/clustalo/)

FIG. 13 provides a schematic view of the different domains for human Rspondin-1.

FIG. 14: Min6 cells were treated with different concentrations of human recombinant Rspo1 (hR1) for 24 hours. Quantification of Min6 revealed that hR1 is able to significantly stimulate immortalized mouse P-cells at a concentration of 200 nM and 400 nM.

FIG. 15: Recombinant hR1 was purified from endotoxin and incubated at different concentration with Min6 cells. After 24 hours, the number of Min6 cells was significantly higher upon exposition with 400 nM and 1 µM of hR1 as compared to controls.

FIG. 16: Quantitative analyses demonstrated that a single dose of hR1 is able to significantly increase the number of proliferative P-cells in WT mice.

FIG. 17: Quantitative studies of immunostained P-cells demostrated that long-term treatment with hR1 significantly increase the number of proliferative P-cells and overall islet size as compared as PBS-injected controls.

### EXAMPLES

### Detailed Protocol for Determining the Activities of Functional Equivalents of Rspondin1 Native Protein

### 1. Binding Affinity to LGR4 Receptor as Determined by SPR Assay

Surface plasmon resonance (SPR) is performed using a Biacore 3000 instrument (Biacore, Uppsala, Sweden). The immobilization of the ligand (mouse Lgr4) is achieved by the activation of dextran coated CM5 chip, followed by covalent bonding of the ligands of the chip surface. Following ligand stabilization, purified Rspo1-recombinant protein (100mM) is allowed to flow over the immobilized-ligand surface and the binding response of analyte to ligand is recorded. The level of interaction will be expressed in response unit (RU), where the maximum value corresponds at the maximum level of affinity/interaction.

### 2. Induction of the Proliferation of Functional Beta Cells as Determined in an In Vitro Beta Cell Proliferation Assay

For proliferation assays upon Rspo1-recombinant protein treatment, cells are seeded into 6-weel plates on glass and on coverslips in a concentration of 150.000 cells/well and maintained in serum-free standard culture medium (supplemented with 1% penicillin/streptomycin) 12 hours before treatment. Cells are cultured for additional 5 min, 1h, 6h and 24h with serum-free standard culture medium containing 67 ng/ml of R1 or medium alone (controls). After treatment, coverslips are first washed in PBS, then fixed for 5 min in 4% PFA, permeabilized for 10 min in 0,1% Triton and stored in PBS at 4° C. with agitation. Prior to immunolabeling, cells are blocked for 45 min in blocking solution (PBS, 10% FCS) and then incubated O.N. at 4° C. with primary antibody (Ki67 1:50, Dako, M7249). Cells are subsequently washed in PBS (3X5min) and incubated 45 min with secondary antibody (e.g. Donkey anti-Rat IgG Secondary Antibody, Alexa Fluor 488 conjugate, 1:1000). Coverslips are mounted with a mounting medium containing DAPI (Vectashield, H-1200) and processed using ZEISS Axiomanager Z1 Imaging System. Proliferation is quantified counting the number of Ki67+ cells per section, normalized on the total cell number/section.

### 3. Induction of the Proliferation of Functional Beta Cells as Determined in an In Vivo Beta Cell Proliferation Assay

Transgenic mouse lines and 129-SV Wild-Type animals (Charles River) were housed and used according to the guidelines of the Belgian Regulations for Animal Care, with the approval by the local Ethical Committee.

Rspo1-recombinant protein (SinoBiological, 50316-M08S) was dissolved in PBS and administered daily intraperitoneally at a concentration of 400 µg/kg.

To assess cell proliferation upon Rspo1 addition, WT mice are treated with Rspo1 and subsequently with BrdU (1 mg/ml via drinking water) for 7 days prior to examination. Cells that has incorporated BrdU during DNA replication are detected using immunohistochemistry.

For immunohistochemistry tissues are isolated and fixed in 4% PFA for 30 minutes at 4° C., dehydrated, embedded in paraffin and sectioned into 6 µm slides. Sections are rehydrated in decreasing concentration of alcohol (Xilene, 100% ethanol, 80% ethanol, 60% ethanol, 30% ethanol and water), then treated with a blocking buffer (PBS 10% Fetal Calf Serum-FCS) and incubated over-night at 4° C. with primary antibodies. For experiments with mouse Rspo1, the primary antibodies used were the following: guinea pig polyclonal anti-insulin (1/500), mouse monoclonal anti-glucagon (1/500) and mouse fluorescein-conjugated anti-bromodeoxyuridine (BrdU) (1/50). Slides were then incubated with secondary antibodies (used 1/1000) for 45 minutes at room temperature and processed using ZEISS Axiomanager Z1 Imaging System. BrdU counting is assessed manually counting proliferative cells within the islets of Langerhans and normalizing the final number on the total islet surface. All values are reported as mean ± SEM of sets of data of at least 5 animals. Data are analyzed using Prism software (GraphPad) by first determining whether they followed a normal distribution using a D’Agostino-Pearson omnibus normality test. If not, un unpaired/nonparametric Mann-Whitney test was used. Conversely, an unpaired t test (2 groups compared) or an unpaired Anova test (more than 2 groups compared) are used assuming Gaussian distribution. Results are considered significant if p < 0.0001 (****), p < 0.001 (***), p < 0.01 (**), and p < 0.05 (*).

### 4. Increase of Glucose-Stimulated Insulin Secretion (GSIS) as Determined in an In Vitro Beta Cell Proliferation Assay

For the evaluation of GSIS upon Rspo1-recombinant protein supplementation, MIN6 cells are incubated for 2h with low (2 mM) and high glucose (25 mM) serum-free standard culture medium and then treated for additional 2h with serum-free standard culture medium containing Rspo1 (67 ng/ml) or medium alone (controls). The medium is then collected and spun down at 2000 X g at 4° C. for 3 min, the supernatant collected and stored at -20° C.

Insulin concentrations from MIN6 supernatants are assessed by ELISA immunoassay (Mercodia, Uppsala, Sweden), following manufactures’ instructions. All reagents and samples were allowed to warm to room temperature before use. Absorbance is read at 450 nm, using a spectrophotometer (Sunrise BasicTecan, Crailsheim, Germany), complemented by a Tecan’s Magellan data analysis software. Insulin concentration was calculated using a second-grade equation on Microsoft Excel. A calibration curve is calculated by plotting the known absorbance value of each Calibrator (except Calibrator 0), against the average of the corresponding insulin concentration value.

### 5. Increase of Glucose-Stimulated Insulin Secretion (GSIS) as Determined in an In Vivo Beta Cell Proliferation Assay

Transgenic mouse lines and 129-SV Wild-Type animals (Charles River) were housed and used according to the guidelines of the Belgian Regulations for Animal Care, with the approval by the local Ethical Committee. Murine Rspo1-recombinant protein was obtained from SinoBiological (50316-M08S).

Rspo1-recombinant protein is dissolved in PBS and administered daily intraperitoneally at a concentration of 400 µg/kg for 5 weeks. For insulinemia measurement, mice are anesthetized using isoflurane delivered in oxygen at a flow rate of 1 l/min. Whole blood samples are collected from the retro-orbital sinus into K3EDTA blood collection tubes, using glass capillaries. In order to measure basal insulinemia, blood samples are drawn after 6 hours of starvation. To assess glucose-stimulated insulin secretion level, an additional blood sampling is performed 2 minutes after an intraperitoneal injection of 2 g/kg of bodyweight of D-(+)-glucose. Whole-blood samples are cooled at once in iced water. Plasma is separated by centrifuging at 2000 X g for 7 minutes at 4C°. The obtained plasma is transferred into pre-cooled tubes, promptly frozen in liquid nitrogen and finally stored at -80C°.

Insulin concentrations from mouse plasma samples are assessed by ELISA immunoassay (Mercodia, Uppsala, Sweden), following manufactures’ instructions. All reagents and samples are allowed to warm to room temperature before use. Absorbance was read at 450 nm, using a spectrophotometer (Sunrise BasicTecan, Crailsheim, Germany), complemented by a Tecan’s Magellan data analysis software. Insulin concentration is calculated using a second-grade equation on Microsoft Excel. A calibration curve is calculated by plotting the known absorbance value of each Calibrator (except Calibrator 0), against the average of the corresponding insulin concentration value.

### Materials and Methods

### Cell Culture

Min6 cells were maintained in Dulbecco’s modified Eagle’s medium (DMEM), containing 25 mmol/L glucose supplemented with 15% heat-inactivated fetal bovine serum, 100 U/ml penicillin, 100 µg/ml streptomycin and 100 µg/ml L-glutamine in humidified 5% CO2,95% air at 37° C. For proliferation assays upon R1 treatment, cells were seeded into 6-weel plates at a concentration of 150.000 cells/well and incubated for 24 h with different concentrations of the molecule tested. Subsequently, cells were washed with phosphate buffered saline (PBS), lifted off the plates with trypsin-EDTA and manually quantified with TOMA chamber.

### Animal Maintenance and Manipulation

Mouse protocols were reviewed and approved by Institutional Ethical committee (Ciepal-Azur) at the university of Nice and all colonies were maintained following European animal research guidelines. Transgenic mouse lines and 129-SV Wild-Type animals (Charles River) were housed and used according to the guidelines of the Belgian Regulations for Animal Care, with the approval by the local Ethical Committee.

Rspo1-recombinant proteins (SinoBiological, 50316-M08S; Peprotech, 120-38) was dissolved in PBS and administered intraperitoneally. To assess cell proliferation upon Rspo1 addition, WT mice were treated with Rspo1 and subsequently with BrdU (1 mg/ml via drinking water) for 7 days prior to examination. Cells that had incorporated BrdU during DNA replication were detected using immunohistochemistry.

### Immunohistochemistry

Tissues were isolated and fixed in 4% PFA for 30 minutes at 4° C., dehydrated, embedded in paraffin and sectioned into 6 µm slides. Sections were rehydrated in decreasing concentration of alcohol (Xilene, 100% ethanol, 80% ethanol, 60% ethanol, 30% ethanol and water), then treated with a blocking buffer (PBS 10% Fetal Calf Serum-FCS) and incubated over-night at 4° C. with primary antibodies. The primary antibodies used were the following guinea pig polyclonal anti-insulin (1/500), mouse monoclonal anti-glucagon (1/500), goat monoclonal anti-somatostatin 1/250), rabbit monoclonal anti-amylase (1/100), rat Ki67 (1/50) and mouse fluorescein-conjugated anti-bromodeoxyuridine (BrdU) (1/50). Slides were then incubated with secondary antibodies (used 1/1000) for 45 minutes at room temperature and processed using ZEISS Axiomanager Z1 and Vectra Polaris Automated Quantitative Pathology Imaging System.

In situ RNA detection of Rspo1 (Probe 401991) and Rspo3 (Probe 402011) transcripts was performed using RNAscope (Advanced Cell Diagnostic). Tissue were quickly isolated and fixed in 10% Buffered Formalin for 16 hours, dehydrated, embedded in paraffin and sectioned into 6 µm slides. Sample pretreatment, probe hybridization and signal amplification and detection were carried according to manufactures’ protocol.

### Intraperitoneal Glucose Tolerance Test (IPGTT) and Blood Glucose Levels Measurement

For the IPGTT, mice were starved for 6h and injected intraperitoneally with a weight-dependent dose of D-(+)-glucose (2 g/kg). Blood glucose levels were measured at the indicated time points after glucose administration using a ONETOUCH Verio glucometer (LifeScan).

### Blood Insulin Levels Measurement

For insulinemia measurement, mice were anesthetized using isoflurane delivered in oxygen at a flow rate of 1 l/min. Whole blood samples were collected from the retro-orbital sinus into K3EDTA blood collection tubes, using glass capillaries. In order to measure basal insulinemia, blood samples were drawn after 6 hours of starvation. To assess glucose-stimulated insulin secretion level, an additional blood sampling was performed 2 minutes after an intraperitoneal injection of 2 g/kg of bodyweight of D-(+)-glucose. Whole-blood samples were cooled at once in iced water. Plasma was separated by centrifuging at 2000 g for 7 minutes at 4C°. The obtained plasma was transferred into pre-cooled tubes, promptly frozen in liquid nitrogen and finally stored at -80C°.

### ELISA Immunoassay

Plasma insulin concentration were assessed by ELISA immunoassay (Mercodia, Uppsala, Sweden), following manufactures’ instructions. All reagents and samples were allowed to warm to room temperature before use. Absorbance was read at 450 nm, using a spectrophotometer (Sunrise BasicTecan, Crailsheim, Germany), complemented by a Tecan’s Magellan data analysis software. Insulin concentration was calculated using Microsoft Excel. A calibration curve was calculated by plotting the known absorbance value of each Calibrator (except Calibrator 0), against the average of the corresponding insulin concentration value.

### Quantification and Data Analysis

Quantitative analyses were performed using the HALO-Indica Labs module on the entire pancreata of at least 5 mice per genotype and per condition. BrdU counting was assessed manually counting proliferative cells within the islets of Langerhans and normalizing the final number on the total islet surface. All values are reported as mean ± SEM of sets of data of at least 5 animals. Data were analyzed using Prism software (GraphPad) by first determining whether they followed a normal distribution using a D’Agostino-Pearson omnibus normality test. If not, un unpaired/nonparametric Mann-Whitney test was used. Conversely, an unpaired t test (2 groups compared) or an unpaired Anova test (more than 2 groups compared) were used assuming Gaussian distribution. Results are considered significant if p < 0.0001 (****), p < 0.001 (***), p < 0.01 (**), and p < 0.05 (*).

### Induction of Streptozotocin-Mediated Diabetes

To induce hyperglycemia, STZ (Sigma) was dissolved in 0.1 M sodium citrate buffer (pH 4.5), and a single dose was administered intraperitoneally (115 mg/kg) within 10 min of dissolution. Diabetes progression was assessed by monitoring blood glucose levels.

### Results

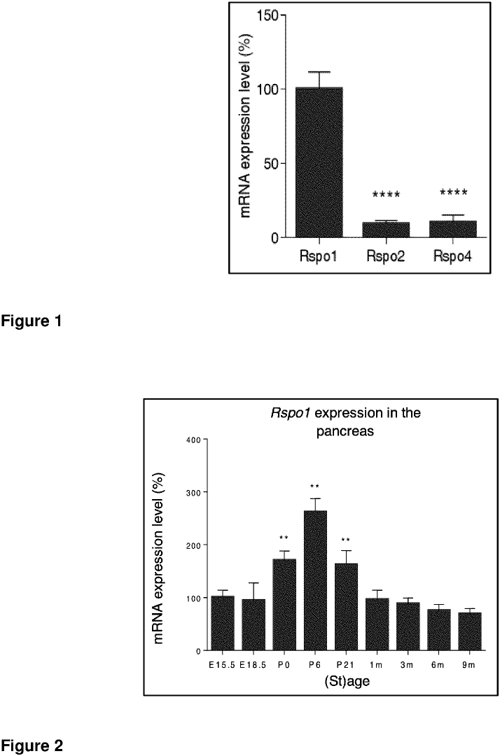

Through a thorough quantitative analysis using RT-qPCR approaches, we demonstrated that Rspo1 is expressed in the pancreas, Rspo2 and Rspo4 mRNAs being not detected at all (FIG. 1). More precisely, Rspo1 is already detectable during embryonic development, starting from embryonic day 15.5 (E15.5). Subsequently, it peaks after birth (around P6) and returns to embryonic development levels during adulthood (FIG. 2)

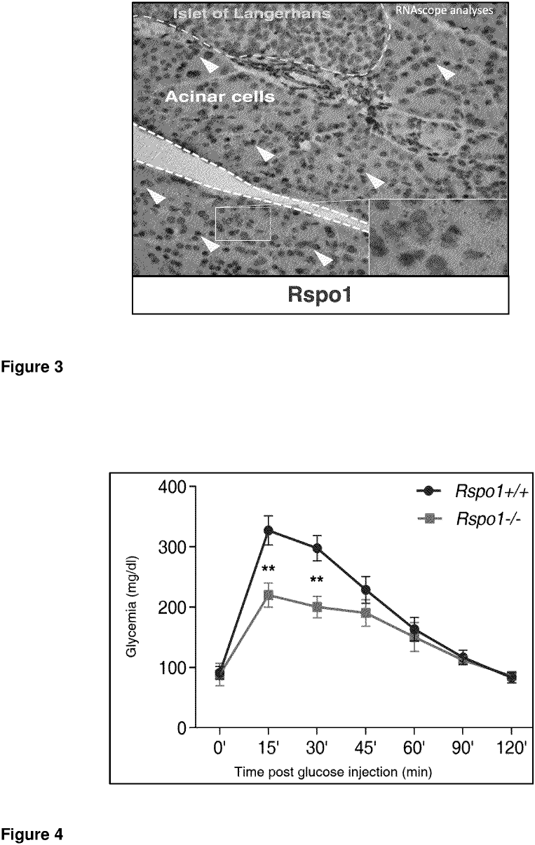

Due to the lack of antibody specifically recognizing Rspo1, we used a relatively novel in situ hybridization technique called RNAscope to assess their localization within the pancreas. The results obtained showed that Rspo1 is located within the exocrine compartment, their expression being restricted to acinar cells (FIG. 3). Interestingly, further analyses outlined an expression of the Rspo1 receptor, Lgr4, solely in the islet of Langerhans and more particularly in beta-cells, the same being seen on human islets (Data not shown).

To assess whether Rspo1 activity is crucial for pancreas development and function, we first analyzed a Rspo1-full knock-out (Rspo1KO) mouse line. In this transgenic line, the targeted disruption of Rspo1 transcripts was achieved by the insertion of a LacZ reporter, followed by a neomycin resistance cassette, into the third exon of the Rspo1 gene (Chassot, A. A. et al. Hum Mol Genet 17, 1264-1277, doi:10.1093/hmg/ddn016 (2008)).

Aiming to determine whether Rspo1 could play a role on pancreatic physiology, despite its expression being confined exclusively to acinar cells, we performed an intra-peritoneal glucose tolerance test (IPGTT), to evaluate the ability of the body to restore normoglycemia upon glucose stimulation. Following a 6-hour starvation period, a weight dependent dose of glucose was dispensed both to Rspo1-/- mutant mice and Rspo1+/+ age-matched controls.

Importantly, mice lacking Rspo1 showed a significantly improved response, with a strong reduction of the glycemic peak. Furthermore, a faster return to euglycemia was also observed in Rspo1-deficient animals (FIG. 4).

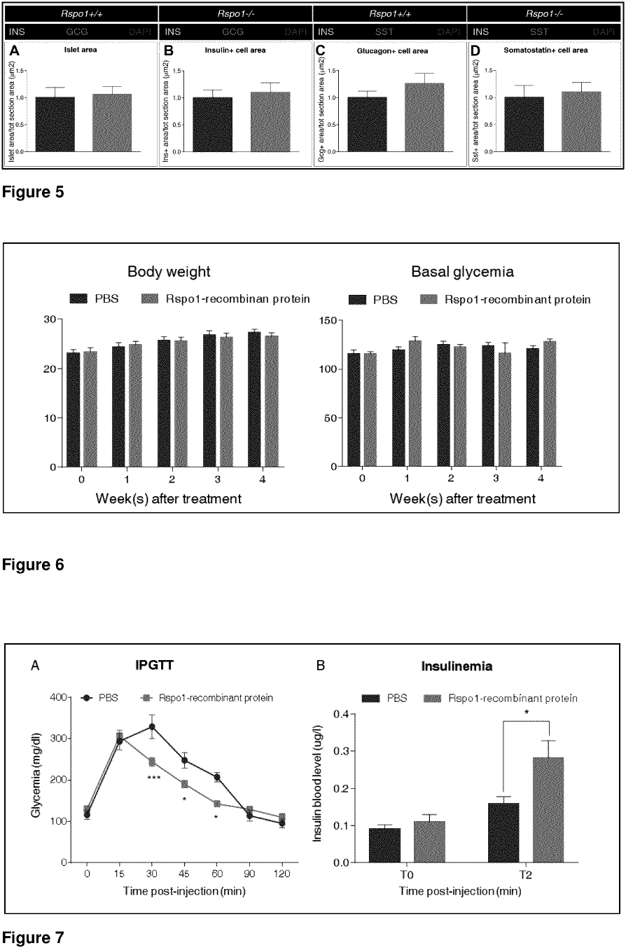

In order to gain further insight into the mechanisms underlying the improved glucose handling in Rspo1-loss-of function animals, we used quantitative analyses. Therefore, pancreatic sections were immuno-stained with antibodies recognizing insulin, glucagon and somatostatin hormones and the stained areas were quantified. A first analysis of the total islet surface revealed no differences between the two groups examined (FIG. 5A). Furthermore, a more detailed quantification did not outline any difference in insulin-(FIG. 5B), glucagon- (FIG. 5C) and somatostatin- (FIG. 5D) expressing cell numbers. Finally, we also assessed the total number of islet per pancreatic section, showing again no discrepancies between Rspo1-/- mice and their age-matched controls (data not shown).

Considering the obtained results, we hypothesized that the ameliorated glucose tolerance upon Rspo1 loss might be caused by peripheral alterations in insulin sensitivity, Rspo1 being genetically removed in all the body, rather than by significant changes in pancreatic cells count and function.

We then wondered about the consequences of over-expression of Rspo1. To achieve this goal, we intra-peritoneally injected wild-type adult mice with a recombinant form of the Rspo1 protein, daily for 4 weeks. The treated mice did not show any difference in body weight and basal glycemia compared to their age-matched controls (only injected with the same volume of saline) throughout the entire treatment (FIG. 6).

Interestingly, an IPGTT revealed that mice treated with the Rspo1-recombinant protein acquired a significantly improved glucose tolerance, with a reduced glycemic peak and a faster recover of normoglycemia compared to the control group (FIG. 7A). In addition, using an ELISA test to measure insulin blood levels, we could also demonstrate that mice injected with the recombinant Rspo1 displayed an increased glucose-stimulated insulin secretion (GSIS) when compared to the age-matched control animals (FIG. 7B).

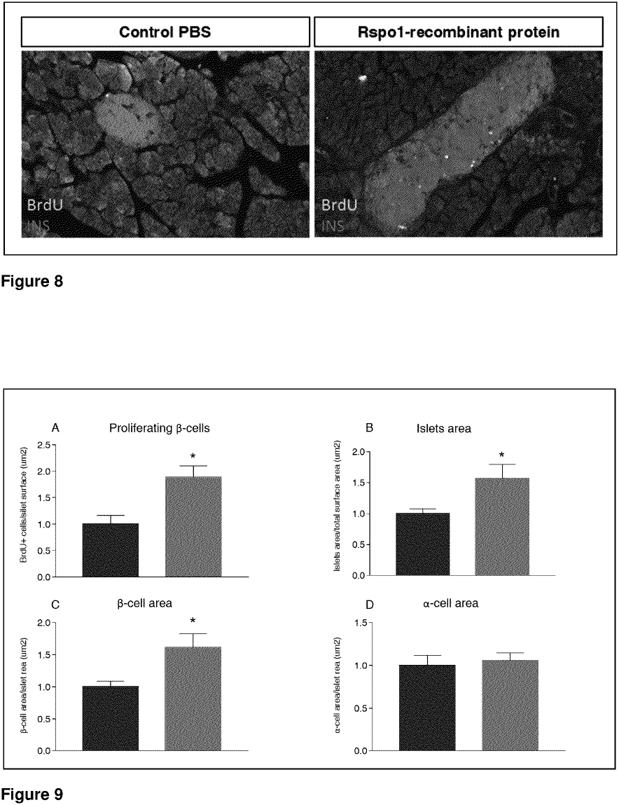

Finally, in order to explain the possible causes of these phenotypes, we resorted to immunofluorescence techniques and stained paraffin pancreatic sections with insulin and BrdU, a marker of cell proliferation. Surprisingly, we not only observed a higher number of proliferating β-cells within the islets of mice treated with Rspo1-recombinant protein, but we also noticed an increased islets size in these mice (FIG. 8). Using quantification analyses, we were able to confirm an increased β-cell proliferation upon Rspo1-recombinant protein injections (FIG. 9A). Consequently, the islets of Langerhans of mice treated with recombinant Rspo1 were found significantly larger compared to age-matched control counterparts only treated with saline (FIG. 9B). This enhanced size is mostly caused by an augmentation of the insulin-producing β-cell mass (FIG. 9C), α-cell mass resulting unchanged after Rspo1 administration (FIG. 9D).

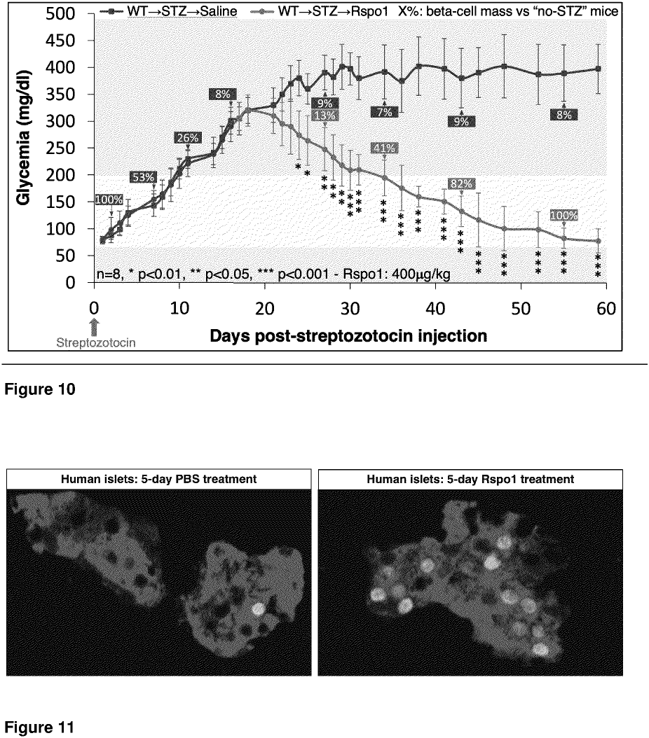

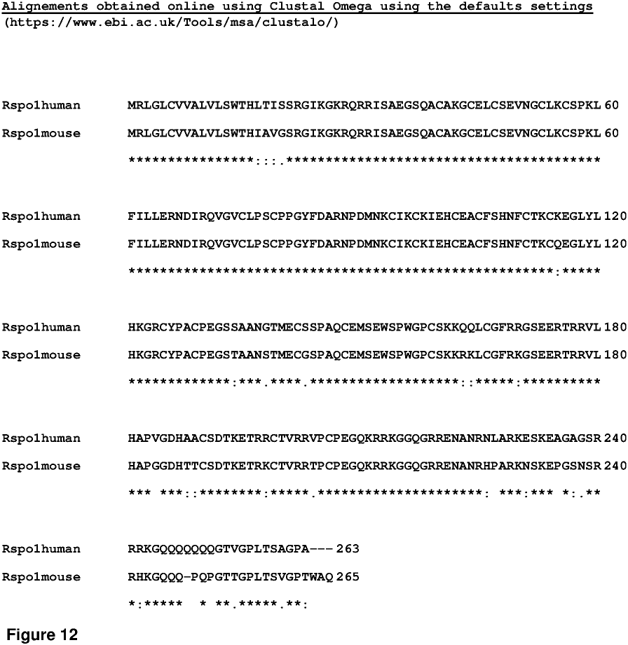

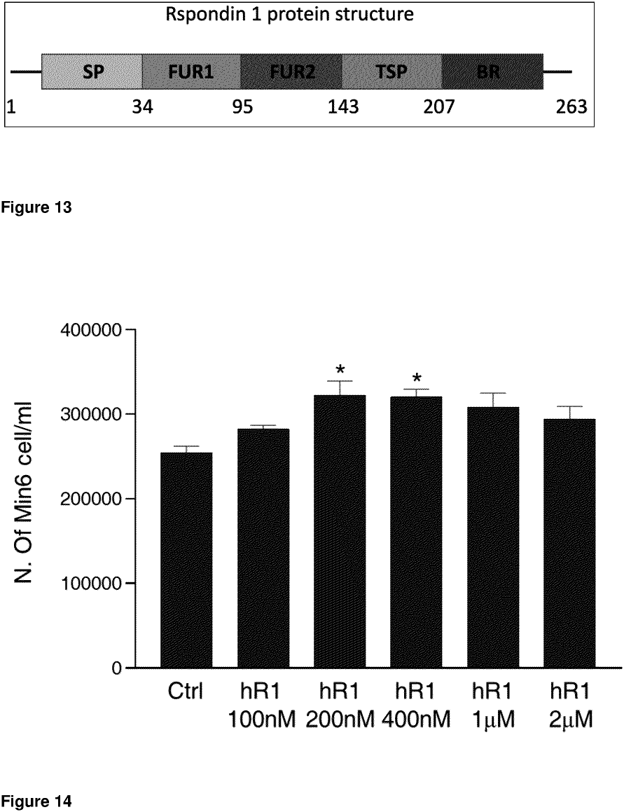

Aiming to determine whether the supplementary insulin-producing cells were functional, WT animals were injected with a high dose of streptozotocin (STZ) to obliterate the pancreatic β-cell mass. Once these animals were overtly diabetic, with a glycemia of approximately 300 mg/dl, they were treated daily with Rspo1 or saline (controls). While saline-treated control mice saw their glycemia increase further, a steady recovery was observed (following a transitory peak in glycemia) in their Rspo1-treated counterparts (FIG. 10). Quantitative immunohistochemical analyses were performed on sections of saline-treated and Rspo1-treated pancreata isolated. While STZ treatment induced a loss of insulin-producing cells in all conditions, animals that received Rspo1 displayed a progressive regeneration of their beta-cell mass, resulting in reconstituted islets following beta-cell ablation (FIG. 10). It is worth noting that weight (data not shown) and glycemia were normal in the surviving animals that displayed an extended life span compared to controls.

Lastly, to determine whether our results could also be translated to human, we cultured human islets in RPMI in presence of Rspo1 (75 mM for 5 days) or not in presence of BrdU to label proliferating cells. Immunohistochemical analyses showed very few proliferating insulin-producing cells in controls (FIG. 11). Interestingly, upon Rspo1 treatment, a massive increase in the number of human proliferating beta-cells was outlined, demonstrating that Rspo1 can also induce human beta-cell proliferation.

### Human Rspo 1

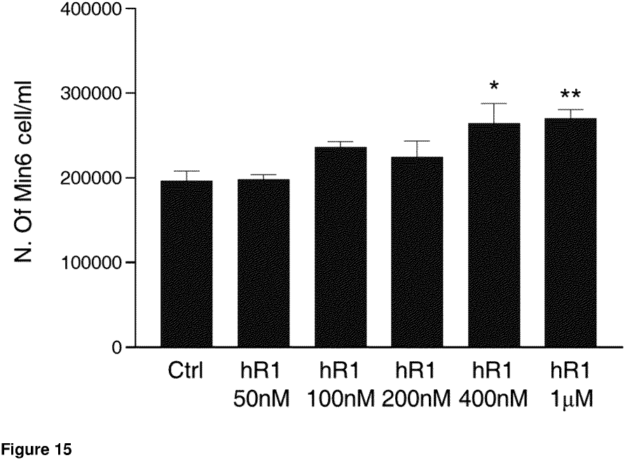

To assess whether human Rspo1 (hR1) was stimulating mouse β-cell proliferation, we incubated hR1 with mouse insulinoma (Min6) cells at different concentrations for 24 hours. Notably, hR1 induced a significant 26% increase in Min6 number as compared as PBS-incubated control cells, at a concentration of 200 nM and 400 nM (FIG. 14). To exclude any contribution of endotoxin to hR1 mitogenicity, we repeated the experiment using an endotoxin purified preparation of hR1. Interestingly, this form of hR1 led to a 35% increase in β-cell number when incubated at a concentration of 400 nM or more (FIG. 15).

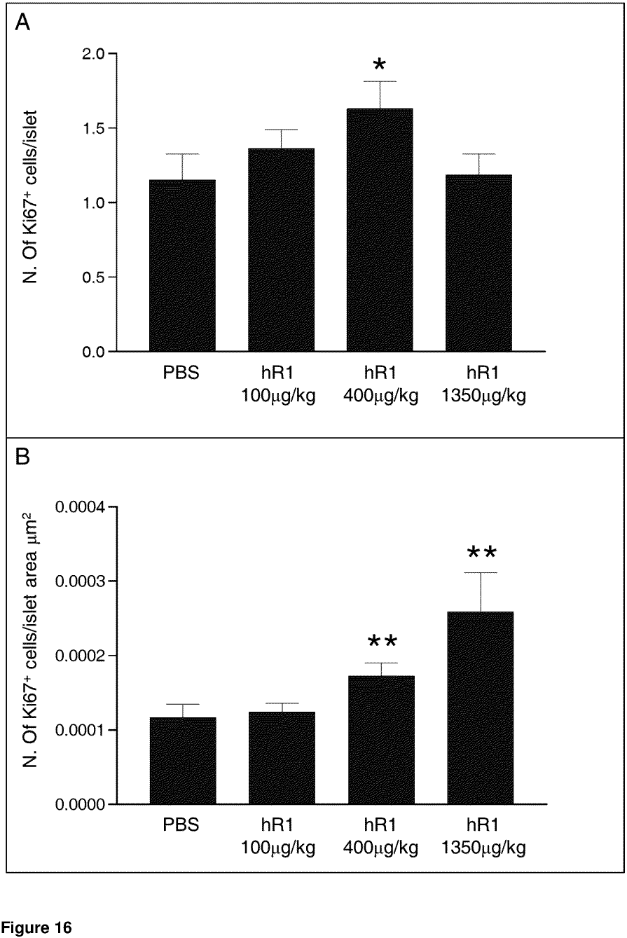

These data clearly show that hR1 exerts a proliferative effect on murine P-cells in vitro. Seeking to transfer our experimental results to in vivo conditions, we performed a short-term treatment on WT rodents. Specifically, mouse pancreata were harvested 30 minutes following injection of hR1 at a concentration of 100 µg/Kg, 400 µg/Kg and 1350 µg/Kg. Immunohistochemical and quantitative analyses of Ki67-labeled P-cells showed that hR1 is able to acutely induce P-cells proliferation when administered in vivo (FIG. 16).

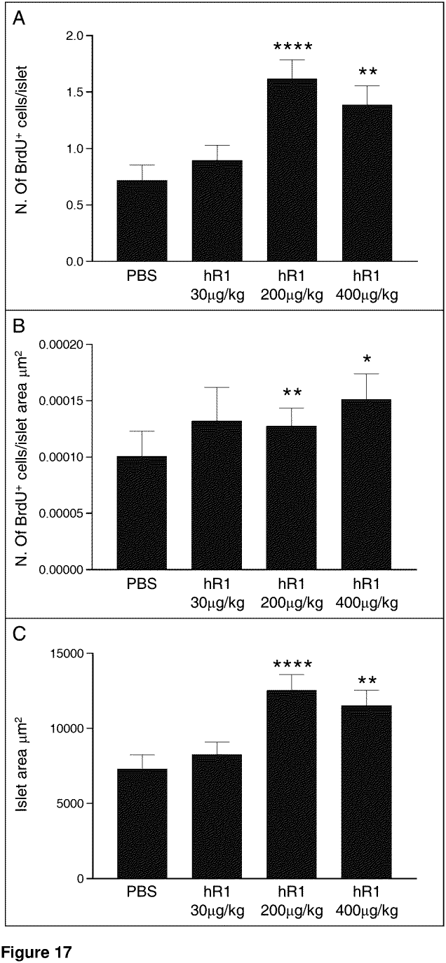

Encouraged by these experimental results, we performed a long-term treatment of adult WT animals daily injected with hR1 at different doses, spanning from 30 µg/Kg to 400 µg/Kg. For this experiment, mice were administered with an endotoxin purified preparation of recombinant hR1 for 28 days. Subsequently, animals were sacrificed and pancreatic tissues were analyzed by immunofluorescence, using antibodies recognizing the β-cell marker prohormone converatese ⅓ (PC1/3) and BrdU to identify proliferating cells. Interestingly, a significant increase in BrdU and PC1/3 double positive cells was observed in mice treated with recombinant hR1 at a concentration of 200 µg/Kg and 400 µg/Kg (FIG. 17). Notably, the hyperproliferation of P-cells was associated with a significant augumentation of pancreatic islets size (FIG. 17).

### Conclusions

The data obtained enlighten a crucial role of Rspo1 in mouse pancreas. Upon the sole overexpression of Rspo1, achieved by daily injections of a recombinant form of the full-length protein, not only the glucose tolerance of the treated mice is significantly ameliorated, but the β-cell mass is increased. Interestingly, this new proliferating β-cells seem also fully functional, being the treated mice able to produce higher amounts of insulin upon glucose stimulation. As important was the finding that upon near complete beta-cell ablation, the remaining beta-cells could be induced to proliferate and reconstitute a functional beta-cells mass able to maintain euglycemia. Our data also strongly indicate that human recombinant Rspo1 is able to stimulate murine P-cells proliferation, increasing the size of the islets of Langerhans. Lastly, the demonstration that Rspo1 can also induce human beta-cell proliferation open new unexpected avenues.

Taken together these results suggest that Rspo1 plays a key paracrine role in the pancreas and that strategies aiming at inscreasing Rspo1 expression, for example by in vivo administration of Rspo1 protein might be beneficial for the treatment and/or the prevention of diabetes in human.

USEFUL SEQUENCES FOR PRACTICING THE INVENTION

| | |
|---|---|
| SEQ ID and Brief description | Sequences |
| SEQ ID NO:1 Human Rspo1 34-143 FU1/2 domains | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECS |
| SEQ ID NO:2 Human Rspo1 34-207 FU1/2 domains + TSP1 | EGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP |
| SEQ ID NO:3 Human Rspo1 34-263 Full-length Rspo1 without SP | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA |
| SEQ ID NO:4 Human Rspo1 isoform 1 (full-length) (NP_001033722.1) | MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA |
| SEQ ID NO:5 Nucleotide Sequence of SEQ ID NO:4 (NM_001038633.4) (coding sequence in bold) | CCCTCTCCGGGCTGGGAGCTCCGGCCGAGCGGAGGCGCGACGGAGAGCACCAGCGCAGGGCAGAGAGCCCGGAGCGACCGGCCAGAGTAGGGCATCCGCTCGGGTGCTGCGGAGAACGAGGGCAGCTCCGAGCCGCCCCGGAGGACCGATGCGCCGGGTGGGGCGCTGGCCCCGAGGGCGTGAGCCGTCCGCAGATTGAGCAACTTGGGAACGGGCGGGCGGAGCGCAGGCGAGCCGGGCGCCCAGGACAGTCCCGCAGCGGGCGGGTGAGCGGGCCGCGCCCTCGCCCCTCCCGGGCCTGCCCCCGTCGCGACTGGCAGCACGAAGCTGAGATTGTGGTTTCCTGGTGATTCAGGTGGGAGTGGGCCAGAAGATCACCGCTGGCAAGGACTGGTGTTTGTCAACTGTAAGGACTCATGGAACAGATCTACCAGGGATTCTCAGACCTTAGTTTGAGAAATGCTGCAATTAAAGGCAAATCCTATCACTCTGAGTGATCGCTTTGGTGTCGAGGCAATCAACCATAAAGATAAATGCAAATATGGAAATTGCATAACAGTACTCAGTATTAAGGTTGGTTTTTGGAGTAGTCCCTGCTGACGTGACAAAAAGATCTCTCATATGATATTCCGAGGTATCTTTGAGGAAGTCTCTCTTTGAGGACCTCCCTTTGAGCTGATGGAGAACTGGGCTCCCCACACCCTCTCTGTCCCCAGCTGAGATTATGGTGGATTTGGGCTACGGCCCAGGCCTGGGCCTCCTGCTGCTGACCCAGCCCCAGAGGTGTTAGCAAGAGCCGTGTGCTATCCACCCTCCCCGAGACCACCCCTCCGACCAGGGGCCTGGAGCTGGCGCGTGACTATGCGGCTTGGGCTGTGTGTGGTGGCCCTGGTTCTGAGCTGGACGCACCTCACCATCAGCAGCCGGGGGATCAAGGGGAAAAGGCAGAGGCGGATCAGTGCCGAGGGGAGCCAGGCCTGTGCCAAAGGCTGTGAGCTCTGCTCTGAAGTCAACGGCTGCCTCAAGTGCTCACCCAAGCTGTTCATCCTGCTGGAGAGGAACGACATCCGCCAGGTGGGCGTCTGCTTGCCGTCCTGCCCACCTGGATACTTCGACGCCCGCAACCCCGACATGAACAAGTGCATCAAATGCAAGATCGAGCACTGTGAGGCCTGCTTCAGCCATAACTTCTGCACCAAGTGTAAGGAGGGCTTGTACCTGCACAAGGGCCGCTGCTATCCAGCTTGTCCCGAGGGCTCCTCAGCTGCCAATGGCACCATGGAGTGCAGTAGTCCTGCGCAATGTGAAATGAGCGAGTGGTCTCCGTGGGGGCCCTGCTCCAAGAAGCAGCAGCTCTGTGGTTTCCGGAGGGGCTCCGAGGAGCGGACACGCAGGGTGCTACATGCCCCTGTGGGGGACCATGCTGCCTGCTCTGACACCAAGGAGACCCGGAGGTGCACAGTGAGGAGAGTGCCGTGTCCTGAGGGGCAGAAGAGGAGGAAGGGAGGCCAGGGCCGGCGGGAGAATGCCAACAGGAACCTGGCCAGGAAGGAGAGCAAGGAGGCGGGTGCTGGCTCTCGAAGACGCAAGGGGCAGCAACAGCAGCAGCAGCAAGGGACAGTGGGGCCACTCACATCTGCAGGGCCTGCCTAGGGACACTGTCCAGCCTCCAGGCCCATGCAGAAAGAGTTCAGTGCTACTCTGCGTGATTCAAGCTTTCCTGAACTGGAACGTCGGGGGCAAAGCATACACACACACTCCAATCCATCCATGCATACATAGACACAAGACACACACGCTCAAACCCCTGTCCACATATACAACCATACATACTTGCACATGTGTGTTCATGTACACACGCAGACACAGACACCACACACACACATACACACACACACACACACACACCTGAGGCCACCAGAAGACACTTCCATCCCTCGGGCCCAGCAGTACACACTTGGTTTCCAGAGCTCCCAGTGGACATGTCAGAGACAACACTTCCCAGCATCTGAGACCAAACTGCAGAGGGGAGCCTTCTGGAGAAGCTGCTGGGATCGGACCAGCCACTGTGGCAGATGGGAGCCAAGCTTGAGGACTGCTGGTGACCTGGGAAGAAACCTTCTTCCCATCCTGTTCAGCACTCCCAGCTGTGTGACTTTATCGTTGGAGAGTATTGTTACCCTTCCAGGATACATATCAGGGTTAACCTGACTTTGAAAACTGCTTAAAGGTTTATTTCAAATTAAAACAAAAAAATCAACGACAGCAGTAGACACAGGCACCACATTCCTTTGCAGGGTGTGAGGGTTTGGCGAGGTATGCGTAGGAGCAAGAAGGGACAGGGAATTTCAAGAGACCCCAAATAGCCTGCTCAGTAGAGGGTCATGCAGACAAGGAAGAAAACTTAGGGGCTGCTCTGACGGTGGTAAACAGGCTGTCTATATCCTTGTTACTCAGAGCATGGCCCGGCAGCAGTGTTGTCACAGGGCAGCTTGTTAGGAATGAGAATCTCAGGTCTCATTCCAGACCTGGTGAGCCAGAGTCTAAATTTTAAGATTCCTGATGATTGGCATGTTACCCAAATTTGAGAAGTGCTGCTGTAATTCCCCTTAAAGGACGGGAGAAAGGGCCCCGGCCATCTTGCAGCAGGAGGGATTCTGGTCAGCTATAAAGGAGGACTTTCCATCTGGGAGAGGCAGAATCTATATACTGAAGGGCTAGTGGCACTGCCAGGGGAAGGGAGTGCGTAGGCTTCCAGTGATGGTTGGGGACAATCCTGCCCAAAGGCAGGGCAGTGGATGGAATAACTCCTTGTGGCATTCTGAAGTGTGTGCCAGGCTCTGGACTAGGTGCTAGGTTTCCAGGGAGGAGCCAAACACGGGCCTTGCTCTTGTGGAGCTTAGAGGTTGGTGGGGAAGAAAATAGGCATGCACCAAGGAATTGTACAAACACATATATAACTACAAAAGGATGGTGCCAAGGGCAGGTGACCACTGGCATCTATGCTTAGCTATGAAAGTGAATAAAGCAGAATAAAAATAAAATACTTTCTCTCAGG |
| SEQ ID NO:6 Mouse amino acid sequence of R-spondin1 (Rspo1) | >NP_619624.2 R-spondin-1 precursor [Mus musculus] MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQ |
| SEQ ID NO:7 Nucleotide sequence of SEQ ID NO:6 >NM_138683.2 (coding sequence in bold) | GGATTCCCTCCCTCGTGCGAGCCGGGGACCGGCCCCTCTCCGGGCGCGGGGCGCAGAGCCCGGGCGGCGCACTGCGGGGCCCGCGCGGGCCGCCCCAGCACCAATGCACCGGGCGGGGCGCTGGCGGCCGAGAAGGCATTGAGCAACTGGGCGGCGGGCGGAGCGCGGGGCCGACGGCAACGCGGGACCCAGTGGCCGCGCCCTCGCCCCTCCGGGCTGCCCCGCCACGGCCGCTGCGCCAGGTCTATCTTGGGGGTGGTTCTCTGCTGGCGTGAGAAGACTTCTCATGTGACCCTCTGAGGTGGATTCAAGCAGGACAGGACCTCCCTTTGGACCAATGGAGAAGCCGGCTCCAAACCCTCTCGGATCCCAGCTAAGGTTATGGTGGATCCGGGCCTGGCTCTCCTGCCACTGACCCAGCCTCAGAGCCTTTTAGCAAGAGACCACCCCTCCTGCCAGGGGCCCGGGGCTGGCCAGTGACTATGCGGCTTGGGCTGTGCGTGGTGGCCCTGGTTCTGAGCTGGACACACATCGCCGTGGGCAGCCGGGGGATCAAGGGCAAGAGACAGAGGCGGATCAGTGCTGAGGGGAGCCAAGCCTGCGCCAAGGGCTGTGAGCTCTGTTCAGAAGTCAACGGTTGCCTCAAGTGCTCGCCCAAGCTCTTCATTCTGCTGGAGAGGAACGACATCCGCCAGGTGGGCGTCTGCCTGCCGTCCTGCCCACCTGGATACTTTGATGCCCGCAACCCCGACATGAACAAATGCATCAAATGCAAGATCGAGCACTGTGAGGCCTGCTTCAGCCACAACTTCTGCACCAAGTGTCAGGAGGGCTTGTACTTACACAAGGGCCGCTGCTATCCAGCCTGCCCTGAGGGCTCTACAGCCGCTAACAGCACCATGGAGTGCGGCAGTCCTGCACAATGTGAAATGAGCGAGTGGTCCCCGTGGGGACCCTGCTCCAAGAAGAGGAAGCTGTGCGGTTTCCGGAAGGGATCGGAAGAGCGGACACGCAGAGTGCTCCATGCTCCCGGGGGAGACCACACCACCTGCTCCGACACCAAAGAGACCCGCAAGTGTACCGTGCGCAGGACGCCCTGCCCAGAGGGGCAGAAGAGGAGGAAGGGGGGCCAGGGCCGGAGGGAGAATGCCAACAGGCATCCGGCCAGGAAGAACAGCAAGGAGCCGGGCTCCAACTCTCGGAGACACAAAGGGCAACAGCAGCCACAGCCAGGGACAACAGGGCCACTCACATCAGTAGGACCTACCTGGGCACAGTGACCGGTCTCCAGATACCTGTGGAAGAGTACAGTGCTGTACT |
| | GTATAATGAGAACTTTCCAGAACTGGAGCATCTGGGAGAGTCCACACATACCCCATCCACCCACCCATCCAACTATCCATCCATCCATCCATGCACACATATGGCCACATCTGAAAACGTCAACACACACACACACACACACACACACACACACACACATTCTTGAGGTCACTGAAGACACTTCTATTCTGTGGCCCAGCTGTATATTCAGTCTTTAATGCTCTTGGAAGACATATCTGAGAGAACCTTTCCCAGCATCTGAAACTAAGGAGTGGAACCTTCTGGAGGAACTTCTGGGACAGCATCTGACAGATGGATGGCAGATTGGAGCCAAAGCTGGAGCAGCTGCCGAGAGGGAGAGAGAGGGAAAGCGCTTTCCCGGCTTGAGAGGCACTCCCAGCTGTGAGACTTGATTGTCGGAGATGAGAATTATTACACATCCGTGGTACACGTCACGGATGACCTGACTTGGAAACTGCTTAAAGGTTTATTTCAAATTAAAAAAGAGAAAAAC |
| SEQ ID NO:8 Human Rspo1 FU2 and TSP1 domains (region from positions 95-207) | IKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP |
| SEQ ID NO:9 Human Rspo1 FU2 and TSP1 and BR domains (region from positions 95-263) | IKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA |
| SEQ ID NO:10 Human Rspo1 FU1+FU2 (region from positions 34-135) | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSA |
| SEQ ID NO:11 Human Rspo1 FU1+FU2 (region from positions 39-132) | ACAKGCELCSEVNGCLKCSPKLFILLERNDIRQVGVCLPSCPPGYFDARNPDMNKCIKCKIEHCEACFSHNFCTKCKEGLYLHKGRCYPACPEG |
| SEQ ID NO:12 Human Rspo1 FU1+FU2 (region from positions 34-143) | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECS |
| SEQ ID NO:13 Human Rspo1 FU1 (region from positions 34-95) | AEGSQACAKGCELCSEVNGCLKCSPKLFILLERNDIRQVGVCLPSCPPGYFDARNPDMNKCI |
| SEQ ID NO:14 Human Rspo1 FU1 (region from positions 34-85) | AEGSQACAKGCELCSEVNGCLKCSPKLFILLERNDIRQVGVCLPSCPPGYFD |
| SEQ ID NO:15 Human Rspo1 FU1 (region from positions 39-86) | ACAKGCELCSEVNGCLKCSPKLFILLERNDIRQVGVCLPSCPPGYFDA |
| SEQ ID NO:16 Human Rspo1 FU2 (region from positions 95-143) | IKCKIEHCEACFSHNFCTKCKEGLYLHKGRCYPACPEGSSAANGTMECS |
| SEQ ID NO:17 Human Rspo1 FU2 | NKCIKCKIEHCEACFSHNFCTKCKEGLYLHKGRCYPACPEG |
| (region from positions 92-132) | |
| SEQ ID NO:18 Human Rspo1 FU2 (region from positions 91-135) | MNKCIKCKIEHCEACFSHNFCTKCKEGLYLHKGRCYPACPEGSSA |
| SEQ ID NO:19 Human Rspo1 FU1 + FU2 + TSP (region from positions 34-206) | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPC |
| SEQ ID NO:20 Human Rspo1 FU1 + FU2 + TSP (region from positions 39-206) | ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPC |
| SEQ ID NO:21 Human Rspo1 FU1 + FU2 + TSP (region from positions 39-207) | ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP |
| SEQ ID NO:22 Human Rspo1 FU1 + FU2 + TSP + BR (region from positions 34-249) | AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQ |
| SEQ ID NO:23 Human Rspo1 FU1 + FU2 + TSP + BR (region from positions 39-263) | ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRR GSEERTRRVLHAPVGDHAACSDTKETRRCTVRRVPCPEGQKRRKGGQGRRENANRNLARKESKEAGAGSRRRKGQQQQQQQGTVGPLTSAGPA |
| SEQ ID NO:24 Human Rspo1 FU1 + FU2 + TSP + BR (region from positions 39-249) | ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQ |
| SEQ ID NO:25 Peptidic linker | GGGGS |

1. A method for treating diabetes in a subject in need thereof comprising administering a therapeutically effective amount of a Rspol protein to said subject.

2. The method of claim 1, wherein said Rspo 1 protein is either:
(i) a protein comprising a R-spondin 1 polypeptide,
(ii) a protein comprising a functional fragment of R-spondin 1 polypeptide, or
(iii) a protein comprising a functional variant of R-spondin 1 polypeptide.

3. The method of claim 1, wherein said Rspo 1 protein is either:
(i) a protein comprising a human R-spondin 1 polypeptide of any one of SEQ ID NOs:2-4,
(ii) a protein comprising a functional fragment of human R-spondin 1 polypeptide of any one of SEQ ID NOs:2-4, or
(iii) a protein comprising a functional variant of R-spondin 1 polypeptide of any one of SEQ ID NOs:2-4.

4. The method of claim 1, wherein said Rspo 1 protein is a protein comprising a functional fragment of R-spondin 1 polypeptide, and wherein said functional fragment comprises or consists of a polypeptide having at least 40-100 consecutive amino acid residues in the FU1 and/or FU2 domains of R-spondin 1 protein.

5. The method of claim 1, wherein said Rspo 1 protein is a recombinant protein comprising either:
(i) any one of SEQ ID NO: 1-4 and SEQ ID NO:8-24, or
(ii) a combination of fragments of Rspol protein of SEQ ID NO:1, including the functional domain FU1 and the functional domain FU2.

6. The method of claim 1, wherein said Rspo 1 protein binds to LGR4 receptor.

7. The method of claim 1, wherein said Rspol protein is a protein comprising a functional fragment or functional variant of a native R-spondin 1 polypeptide and said Rspol protein exhibits at least 50%, 60%, 70%, 80%, 90%, or 100% of one or more of the following activities relative to said native R-spondin 1:
i. Binding affinity to LGR4 receptor determined by SPR assay;
ii. Induction of the proliferation of functional beta cells determined in an in vitro beta cell proliferation assay;
iii. Induction of the proliferation of functional beta cells determined in an in vivo beta cell proliferation assay;
iv. Increase of glucose-stimulated insulin secretion (GSIS) determined in an in vitro beta cell proliferation assay; or,
v. Increase of glucose-stimulated insulin secretion (GSIS) determined in an in vivo beta cell proliferation assay.

8. The method of claim 1, wherein said Rspo 1 protein is a protein comprising a functional variant of R-spondin 1, wherein said functional variant comprises or essentially consists of a polypeptide having at least 70%, 80%, 90% or at least 95% identity to a parent R-spondin 1 polypeptide sequence.

9. The method of claim 8, wherein said functional variant of R-spondin 1 differs from the corresponding native R-spondin 1 sequence through only amino acid substitutions.

10. The method of claim 1, wherein said Rspo 1 protein is a fusion protein.

11. The method of claim 1, wherein said Rspo 1 protein is a pegylated or PASylated protein.

12. The method of claim 1, wherein said diabete is diabete type 1 or type 2.

13. The method of claim 1, wherein said therapeutically effective amount of Rspol protein is administered via the subcutaneous or intravenous route to the subject.

14. The method of claim 1, wherein said subject is a human subject.

15. A pharmaceutical composition comprising the Rspol protein as defined in claim 1, and one or more pharmaceutically acceptable excipients.

16. The method of claim 4, wherein said functional fragment comprises or consists of a polypeptide having at least 40-100 consecutive amino acid residues in the FU1 and/or FU2 domains of any of the polypeptides of SEQ ID NO: 1-4 and SEQ ID NO:8-24.

17. The method of claim 5, wherein said Rsp1 protein is a recombinant protein comprising a combination of fragments of Rspo1 protein of SEQ ID NO:1 including the functional domain FU1 and the functional domain FU2 and the functional domain TSP.

18. The method of claim 1, wherein said Rspol protein is a protein comprising a functional variant of R-spondin 1, wherein said functional variant comprises or essentially consists of a polypeptide having at least 70%, 80%, 90% or at least 95% identity to one of polypeptides of SEQ ID NOs :1-4 and SEQ ID NO :8-24.

19. The method of claim 1, wherein said Rspol protein is a fusion protein comprising an Fc region of an antibody.


SEQUENCE LISTING (25 sequences)

SEQ ID NO: 1 - protein, 110 aa, Homo sapiens
AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECS

SEQ ID NO: 2 - protein, 173 aa, Homo sapiens
EGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP

SEQ ID NO: 3 - protein, 230 aa, Homo sapiens
AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 4 - protein, 263 aa, Homo sapiens
MRLGLCVVALVLSWTHLTISSRGIKGKRQRRISAEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 5 - DNA, 3058 bp, Homo sapiens
ccctctccgggctgggagctccggccgagcggaggcgcgacggagagcaccagcgcagggcagagagcccggagcgaccggccagagtagggcatccgctcgggtgctgcggagaacgagggcagctccgagccgccccggaggaccgatgcgccgggtggggcgctggccccgagggcgtgagccgtccgcagattgagcaacttgggaacgggcgggcggagcgcaggcgagccgggcgcccaggacagtcccgcagcgggcgggtgagcgggccgcgccctcgcccctcccgggcctgcccccgtcgcgactggcagcacgaagctgagattgtggtttcctggtgattcaggtgggagtgggccagaagatcaccgctggcaaggactggtgtttgtcaactgtaaggactcatggaacagatctaccagggattctcagaccttagtttgagaaatgctgcaattaaaggcaaatcctatcactctgagtgatcgctttggtgtcgaggcaatcaaccataaagataaatgcaaatatggaaattgcataacagtactcagtattaaggttggtttttggagtagtccctgctgacgtgacaaaaagatctctcatatgatattccgaggtatctttgaggaagtctctctttgaggacctccctttgagctgatggagaactgggctccccacaccctctctgtccccagctgagattatggtggatttgggctacggcccaggcctgggcctcctgctgctgacccagccccagaggtgttagcaagagccgtgtgctatccaccctccccgagaccacccctccgaccaggggcctggagctggcgcgtgactatgcggcttgggctgtgtgtggtggccctggttctgagctggacgcacctcaccatcagcagccgggggatcaaggggaaaaggcagaggcggatcagtgccgaggggagccaggcctgtgccaaaggctgtgagctctgctctgaagtcaacggctgcctcaagtgctcacccaagctgttcatcctgctggagaggaacgacatccgccaggtgggcgtctgcttgccgtcctgcccacctggatacttcgacgcccgcaaccccgacatgaacaagtgcatcaaatgcaagatcgagcactgtgaggcctgcttcagccataacttctgcaccaagtgtaaggagggcttgtacctgcacaagggccgctgctatccagcttgtcccgagggctcctcagctgccaatggcaccatggagtgcagtagtcctgcgcaatgtgaaatgagcgagtggtctccgtgggggccctgctccaagaagcagcagctctgtggtttccggaggggctccgaggagcggacacgcagggtgctacatgcccctgtgggggaccatgctgcctgctctgacaccaaggagacccggaggtgcacagtgaggagagtgccgtgtcctgaggggcagaagaggaggaagggaggccagggccggcgggagaatgccaacaggaacctggccaggaaggagagcaaggaggcgggtgctggctctcgaagacgcaaggggcagcaacagcagcagcagcaagggacagtggggccactcacatctgcagggcctgcctagggacactgtccagcctccaggcccatgcagaaagagttcagtgctactctgcgtgattcaagctttcctgaactggaacgtcgggggcaaagcatacacacacactccaatccatccatgcatacatagacacaagacacacacgctcaaacccctgtccacatatacaaccatacatacttgcacatgtgtgttcatgtacacacgcagacacagacaccacacacacacatacacacacacacacacacacacctgaggccaccagaagacacttccatccctcgggcccagcagtacacacttggtttccagagctcccagtggacatgtcagagacaacacttcccagcatctgagaccaaactgcagaggggagccttctggagaagctgctgggatcggaccagccactgtggcagatgggagccaagcttgaggactgctggtgacctgggaagaaaccttcttcccatcctgttcagcactcccagctgtgtgactttatcgttggagagtattgttacccttccaggatacatatcagggttaacctgactttgaaaactgcttaaaggtttatttcaaattaaaacaaaaaaatcaacgacagcagtagacacaggcaccacattcctttgcagggtgtgagggtttggcgaggtatgcgtaggagcaagaagggacagggaatttcaagagaccccaaatagcctgctcagtagagggtcatgcagacaaggaagaaaacttaggggctgctctgacggtggtaaacaggctgtctatatccttgttactcagagcatggcccggcagcagtgttgtcacagggcagcttgttaggaatgagaatctcaggtctcattccagacctggtgagccagagtctaaattttaagattcctgatgattggcatgttacccaaatttgagaagtgctgctgtaattccccttaaaggacgggagaaagggccccggccatcttgcagcaggagggattctggtcagctataaaggaggactttccatctgggagaggcagaatctatatactgaagggctagtggcactgccaggggaagggagtgcgtaggcttccagtgatggttggggacaatcctgcccaaaggcagggcagtggatggaataactccttgtggcattctgaagtgtgtgccaggctctggactaggtgctaggtttccagggaggagccaaacacgggccttgctcttgtggagcttagaggttggtggggaagaaaataggcatgcaccaaggaattgtacaaacacatatataactacaaaaggatggtgccaagggcaggtgaccactggcatctatgcttagctatgaaagtgaataaagcagaataaaaataaaatactttctctcagg

SEQ ID NO: 6 - protein, 265 aa, Mus musculus
MRLGLCVVALVLSWTHIAVGSRGIKGKRQRRISAEGSQACAKGCELCSEVNGCLKCSPKLFILLERNDIRQVGVCLPSCPPGYFDARNPDMNKCIKCKIEHCEACFSHNFCTKCQEGLYLHKGRCYPACPEGSTAANSTMECGSPAQCEMSEWSPWGPCSKKRKLCGFRKGSEERTRRVLHAPGGDHTTCSDTKETRKCTVRRTPCPEGQKRRKGGQGRRENANRHPARKNSKEPGSNSRRHKGQQQPQPGTTGPLTSVGPTWAQ

SEQ ID NO: 7 - DNA, 1834 bp, Mus musculus
ggattccctccctcgtgcgagccggggaccggcccctctccgggcgcggggcgcagagcccgggcggcgcactgcggggcccgcgcgggccgccccagcaccaatgcaccgggcggggcgctggcggccgagaaggcattgagcaactgggcggcgggcggagcgcggggccgacggcaacgcgggacccagtggccgcgccctcgcccctccgggctgccccgccacggccgctgcgccaggtctatcttgggggtggttctctgctggcgtgagaagacttctcatgtgaccctctgaggtggattcaagcaggacaggacctccctttggaccaatggagaagccggctccaaaccctctcggatcccagctaaggttatggtggatccgggcctggctctcctgccactgacccagcctcagagccttttagcaagagaccacccctcctgccaggggcccggggctggccagtgactatgcggcttgggctgtgcgtggtggccctggttctgagctggacacacatcgccgtgggcagccgggggatcaagggcaagagacagaggcggatcagtgctgaggggagccaagcctgcgccaagggctgtgagctctgttcagaagtcaacggttgcctcaagtgctcgcccaagctcttcattctgctggagaggaacgacatccgccaggtgggcgtctgcctgccgtcctgcccacctggatactttgatgcccgcaaccccgacatgaacaaatgcatcaaatgcaagatcgagcactgtgaggcctgcttcagccacaacttctgcaccaagtgtcaggagggcttgtacttacacaagggccgctgctatccagcctgccctgagggctctacagccgctaacagcaccatggagtgcggcagtcctgcacaatgtgaaatgagcgagtggtccccgtggggaccctgctccaagaagaggaagctgtgcggtttccggaagggatcggaagagcggacacgcagagtgctccatgctcccgggggagaccacaccacctgctccgacaccaaagagacccgcaagtgtaccgtgcgcaggacgccctgcccagaggggcagaagaggaggaaggggggccagggccggagggagaatgccaacaggcatccggccaggaagaacagcaaggagccgggctccaactctcggagacacaaagggcaacagcagccacagccagggacaacagggccactcacatcagtaggacctacctgggcacagtgaccggtctccagatacctgtggaagagtacagtgctgtactgtataatgagaactttccagaactggagcatctgggagagtccacacataccccatccacccacccatccaactatccatccatccatccatgcacacatatggccacatctgaaaacgtcaacacacacacacacacacacacacacacacacacacattcttgaggtcactgaagacacttctattctgtggcccagctgtatattcagtctttaatgctcttggaagacatatctgagagaacctttcccagcatctgaaactaaggagtggaaccttctggaggaacttctgggacagcatctgacagatggatggcagattggagccaaagctggagcagctgccgagagggagagagagggaaagcgctttcccggcttgagaggcactcccagctgtgagacttgattgtcggagatgagaattattacacatccgtggtacacgtcacggatgacctgacttggaaactgcttaaaggtttatttcaaattaaaaaagagaaaaac

SEQ ID NO: 8 - protein, 113 aa, Homo sapiens
IKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP

SEQ ID NO: 9 - protein, 169 aa, Homo sapiens
IKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 10 - protein, 102 aa, Homo sapiens
AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSA

SEQ ID NO: 11 - protein, 94 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEG

SEQ ID NO: 12 - protein, 110 aa, Homo sapiens; identical to SEQ ID NO: 1

SEQ ID NO: 13 - protein, 62 aa, Homo sapiens
AEGSQACAKGCELCSEVNGCLKCSPKLFILLERNDIRQVGVCLPSCPPGYFDARNPDMNKCI

SEQ ID NO: 14 - protein, 52 aa, Homo sapiens
AEGSQACAKGCELCSEVNGCLKCSPKLFILLERNDIRQVGVCLPSCPPGYFD

SEQ ID NO: 15 - protein, 48 aa, Homo sapiens
ACAKGCELCSEVNGCLKCSPKLFILLERNDIRQVGVCLPSCPPGYFDA

SEQ ID NO: 16 - protein, 49 aa, Homo sapiens
IKCKIEHCEACFSHNFCTKCKEGLYLHKGRCYPACPEGSSAANGTMECS

SEQ ID NO: 17 - protein, 41 aa, Homo sapiens
NKCIKCKIEHCEACFSHNFCTKCKEGLYLHKGRCYPACPEG

SEQ ID NO: 18 - protein, 45 aa, Homo sapiens
MNKCIKCKIEHCEACFSHNFCTKCKEGLYLHKGRCYPACPEGSSA

SEQ ID NO: 19 - protein, 173 aa, Homo sapiens
AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPC

SEQ ID NO: 20 - protein, 168 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPC

SEQ ID NO: 21 - protein, 169 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCP

SEQ ID NO: 22 - protein, 216 aa, Homo sapiens
AEGSQACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQ

SEQ ID NO: 23 - protein, 225 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQQQGTVGPLTSAGPA

SEQ ID NO: 24 - protein, 211 aa, Homo sapiens
ACAKGCELCSEVNGCLKCSPKLFILLERNDIRQVGVCLPSCPPGYFDARNPDMNKCIKCKIEHCEACFSHNFCTKCKEGLYLHKGRCYPACPEGSSAANGTMECSSPAQCEMSEWSPWGPCSKKQQLCGFRRGSEERTRRVLHAPVGDHAACSDTKETRRCTVRRVPCPEGQKRRKGGQGRRENANRNLARKESKEAGAGSRRRKGQQQQQ

SEQ ID NO: 25 - protein, 5 aa, Artificial Sequence
  Linker
GGGGS